(19) United States
(12) Patent Application Publication
(10) Pub. No.: US 2020/0390823 A1
(43) Pub. Date: Dec. 17, 2020

(54) FREE-STANDING, TRANSFERRABLE VASCULAR NETWORKS VIA GUIDED SELF-ASSEMBLY AND USE IN CELL REPLACEMENT THERAPIES

(71) Applicant: CORNELL UNIVERSITY, Ithaca, NY (US)

(72) Inventors: Minglin MA, Ithaca, NY (US); Wei SONG, Ithaca, NY (US); Alan CHIU, Providence, RI (US)

(21) Appl. No.: 16/968,392

(22) PCT Filed: Feb. 7, 2019
(86) PCT No.: PCT/US2019/016995

§ 371 Date: Aug. 7, 2020

Related U.S. Application Data
(60) Provisional application No. 62/627,593, filed on Feb. 7, 2018.

(51) Int. Cl. A61K 35/44; A61K 35/39; A61K 9/14

(52) U.S. Cl. CPC A61K 35/44; A61K 9/14; A61K 35/39

(57) ABSTRACT

The present invention relates to a vascularized therapeutic delivery system. This system includes a preparation of cells encapsulated by a biological support material and a microvascular mesh that at least partially surrounds the biological support material encapsulating the preparation of cells, where the microvascular mesh includes a network of continuous interconnected tubular structures defined by endothelial cells and an extracellular matrix scaffold. Also disclosed are methods for delivering a therapeutic agent to a subject using the vascularized therapeutic delivery system and methods of producing such a vascularized therapeutic delivery system.

This application claims the priority benefit of U.S. Provisional Patent Application Ser. No. 62/627,593, filed Feb. 7, 2018, which is hereby incorporated by reference in its entirety.

### FIELD

The present invention relates to vascularized therapeutic delivery systems, methods for delivery of a therapeutic agent to a subject using such vascularized therapeutic delivery systems, and methods of producing vascularized therapeutic delivery systems.

### BACKGROUND

The following description is provided to assist the understanding of the reader. None of the information provided or references cited is admitted to be prior art to the present technology.

Vasculature is an essential component of almost any tissue or organ (Carmeliet, P., “Angiogenesis in Life, Disease and Medicine,” Nature 438:932-936 (2005)) and therefore vascular regeneration is critical to the success of bioengineered implants (Song et al., “Vascular Tissue Engineering: Progress, Challenges, and Clinical Promise,” Cell Stem Cell 22:340-354 (2018); Zhang et al., “Biodegradable Scaffold With Built-In Vasculature for Organ-on-a-Chip Engineering and Direct Surgical Anastomosis,” Nat. Mater. 15:669-678 (2016); Lee et al., “Light-Triggered In Vivo Activation of Adhesive Peptides Regulates Cell Adhesion, Inflammation and Vascularization of Biomaterials,” Nat. Mater. 14:352-360 (2015); McGuigan et al., “Vascularized Organoid Engineered by Modular Assembly Enables Blood Perfusion,” Proc. Natl. Acad. Sci. U.S.A. 103:11461-11466 (2006); and Baranski et al., “Geometric Control of Vascular Networks to Enhance Engineered Tissue Integration and Function,” Proc. Natl. Acad. Sci. U.S.A. 110:7586-7591 (2013)). For example, in cell replacement therapies for type 1 diabetes (T1D), transplanted insulin producing cells rely on nearby vasculature to survive and function (Pepper et al., “A Prevascularized Subcutaneous Device-Less Site for Islet and Cellular Transplantation,” Nat. Biotechnol. 33:518-523 (2015); Vlahos et al., “Modular Tissue Engineering for the Vascularization of Subcutaneously Transplanted Pancreatic Islets,” Proc. Natl. Acad. Sci. U.S.A. 114:9337-9342 (2017); Mahou et al., “Injectable and Inherently Vascularizing Semi-Interpenetrating Polymer Network for Delivering Cells to the Subcutaneous Space,” Biomaterials 131:27-35 (2017); Weaver et al., “Design of a Vascularized Synthetic Poly(ethylene Glycol) Macroencapsulation Device for Islet Transplantation,” Biomaterials 172:54-65 (2018); Weaver et al., “Vasculogenic Hydrogel Enhances Islet Survival, Engraftment, and Function in Leading Extrahepatic Sites,” Sci. Adv. 3:e1700184 (2017); and Phelps et al., “Vasculogenic Bio-Synthetic Hydrogel for Enhancement of Pancreatic Islet Engraftment and Function in Type 1 Diabetes,” Biomaterials 34:4602-4611 (2013).

Vascular endothelial cells such as human umbilical vein endothelial cells (HUVECs) can spontaneously assemble into tubular structures in an extracellular matrix (ECM) such as fibrin, however, the structures are usually random, uncontrollable, and less efficient for promoting microvascular regeneration (Verseij den et al., “Vascularization of Prevascularized and Non-Prevascularized Fibrin-Based Human Adipose Tissue Constructs After Implantation in Nude Mice,” J. Tissue Eng. Regener. Med. 6:169-178 (2012)). While conventional molding methods such as seeding cell/matrix mixtures into grooved templates may guide self-assembly into specific patterns (Jiang et al., “Guided Assembly of Endothelial Cells on Hydrogel Matrices Patterned With Microgrooves: A Basic Model for Microvessel Engineering,” Soft Matter 9:1113-1121 (2013) and Vrij et al., “Directed Assembly and Development of Material-Free Tissues With Complex Architectures,” Adv. Mater. 28:4032-4039 (2016)), such structures tend to shrink and clump during maturation due to intrinsic contractile forces (Wang, H. et al., “Necking and Failure of Constrained 3D Microtissues Induced by Cellular Tension,” Proc. Natl. Acad. Sci. U.S.A. 110:20923-20928 (2013)), making fabrication and scale-up of stable and transferrable vasculature a challenge. Interconnected endothelial lumen structures can also be formed by perfusing endothelial cells in microfluidic channels and lining the cells along channel walls (Zheng, Y. et al., “In Vitro Microvessels for the Study of Angiogenesis and Thrombosis,” Proc. Natl. Acad. Sci. U.S.A. 109:9342-9347 (2012)). However, confinement within channels inevitably precludes the transfer of endothelial structure to other substrates or devices, limiting the scope of its applications in transplantation. Recent developments in 3D printing techniques have provided opportunities to fabricate cellular constructs with embedded vascular structures in a controlled manner (Kolesky, D. B. et al., “3D Bioprinting of Vascularized, Heterogeneous Cell-Laden Tissue Constructs,” Adv. Mater. 26:3124-3130 (2014) and Miller, J. S. et al., “Rapid Casting of Patterned Vascular Networks For Perfusable Engineered Three-Dimensional Tissues,” Nat. Mater. 11:768-774 (2012)). However, it remains challenging to print high resolution, microscale vascular structure that is resilient and transferrable to different substrates for transplantation applications.

The present application is directed to overcoming these and other deficiencies in the art.

### SUMMARY

A first aspect of the present invention relates to a vascularized therapeutic delivery system. This system includes a preparation of cells encapsulated by a biological support material and a microvascular mesh that at least partially surrounds the biological support material encapsulating the preparation of cells. The microvascular mesh includes a network of continuous interconnected tubular structures defined by endothelial cells and an extracellular matrix scaffold.

Another aspect of the present invention is directed to a method for delivering a therapeutic agent to a subject. This method involves selecting a subject in need of a therapeutic agent; providing the vascularized therapeutic delivery system described herein; and implanting the vascularized therapeutic delivery system into a region of the selected subject suitable for delivering the therapeutic agent.

A further aspect of the present invention is directed to a method of producing a vascularized therapeutic delivery system. This method involves providing a monolithic substrate comprising a planar surface interrupted by a plurality of micropillars, and applying endothelial cells suspended in an extracellular matrix material to the planar surface of the monolithic substrate. The method further involves culturing the suspended endothelial cells under conditions effective for the endothelial cells to organize into a microvascular mesh, where the microvascular mesh comprises a network of continuous interconnected tubular structures defined by the endothelial cells and extracellular matrix material. The method further involves transferring the microvascular mesh to an outer surface of an implantable cell delivery construct comprising a preparation of cells, thereby producing the vascularized therapeutic delivery system.

The present application provides a micropillar-based, Anchored Self-Assembly (ASA) strategy to fabricate controllable, transferrable, and scalable microvascular meshes for application in cell replacement therapies and therapeutic delivery. In the ASA, micropillars guide self-assembly of endothelial cells within a fibrin matrix while serving as anchoring points to prevent cellular structure from shrinkage during vessel maturation, leading to controllable, interconnected, and resilient fibrin-filled tubular structures (i.e., “microvascular mesh”). Tuning the dimension and arrangement of micropillars allows for the control of the geometry (square, pentagon, hexagon, octagon, spider web-like, microcapillary-like), diameter of the fibrin-filled tubes, and mesh opening of the microvascular mesh. These meshes are scalable to centimeter size (e.g., 5×5 cm) and transferable to diverse substrates. Upon transplantation, microvascular meshes promoted both neovascularization and vascular anastomoses. When attached to a diffusion chamber device, the meshes led to the formation of a functional microvascular network with a density as high as ˜220 vessels/mm² around the device within the poorly vascularized subcutaneous space of SCID-Beige mice. The present application further demonstrates the feasibility of engineering patient-specific microvasculature using human induced pluripotent stem cell-derived endothelial cells (iPSC-ECs). The microvascular meshes (both HUVECs and iPSC-ECs) significantly improved vascularization of subcutaneously transplanted rat islets (i.e., a “subcutaneous pancreas”) and HUVEC meshes enabled a 3-month cure of streptozotocin (STZ)-induced diabetes in SCID-Beige mice, providing a proof-of-concept for the translatability of microvascular meshes in cell replacement therapies for T1D and other diseases.

### BRIEF DESCRIPTION OF THE DRAWINGS

FIGS. 1A-1F show the fabrication and function of transferrable microvascular meshes via anchored self-assembly (“ASA”). FIG. 1A is a schematic illustration of organization of vascular endothelial cells into a microvascular mesh which is transferred and attached to a cellular device. In a poorly-vascularized subcutaneous space, microvascular mesh can enhance vascularization and anastomoses with host vasculature to provide oxygen/nutrients to donor cells. FIG. 1B shows the design of micropillar arrangement and ASA-enabled cell organization: the key to the ASA is that inner micropillars provide a geometric template for cell self-assembly to form square mesh and boundary micropillars serve as anchoring points for cell attachment to prevent assembled mesh structure from shrinking. FIG. 1C is a fluorescent image of HUVECs expressing GFP in fibrin matrix organized into a square mesh (1×1 cm) on a micropillar substrate via ASA after 2 days of culture. FIG. 1D shows an image of a HUVEC square mesh (5×5 cm) lifted from the micropillar substrate for transfer after 2 days of culture. The insert is a magnified image of HUVEC mesh and scale bar is 1 mm. FIG. 1E is an image showing that HUVEC microvascular mesh generates angiogenic sprouts (white arrows) when embedded in fibrin matrix during 2 weeks of culture in vitro. FIG. 1F shows microscopic images of retrieved HUVEC microvascular mesh device show a high degree of vascularization after 2 weeks of subcutaneous implantation in SCID-Beige mice (n=4).

FIGS. 2A-2F show the simulation and characterization of the ASA-enabled microvascular meshes. FIGS. 2A-2B show the results of a contraction simulation showing an in-plane displacement contour plot of organized cellular mesh structure (FIG. 2A) and the normal stress distribution in the X (Cauchy stress component 11) direction (FIG. 2B) on a 4×4 micropillar substrate. The initial shape of cells and fibrin matrix is displayed in light gray. The micropillar diameter is 400 μm and micropillar-to-micropillar interval is 200 μm. The contracted region is marked as dotted ellipse and the junction region is a circle. The displacement unit is μm and the unit of stress is mN/μm ². FIG. 2C are cross-sectional images showing a HUVEC mesh suspends between micropillars. The micropillars are pseudo-colored as light grey and HUVECs are pseudo-colored as dark grey. FIG. 2D shows SEM images of a HUVEC mesh at the inner and boundary regions on the micropillar substrate. FIG. 2E shows confocal images of a HUVEC mesh at the contracted and junction regions on the micropillar substrate showing the tubular structures. Human CD31 antibody is green, F-actin is red, and nucleus is blue. FIG. 2F shows screenshots of a glass pipette poking a HUVEC mesh showing high resilience of the mesh.

FIGS. 3A-3I shows the enhancement of vascularization and anastomoses in the subcutaneous space of SCID-Beige mice. FIG. 3A is a schematic illustration and a digital photo of the vascular therapeutic delivery system of the present invention comprising a diffusion chamber device encapsulating cells wrapped with HUVEC microvascular meshes. FIG. 3B shows fluorescent images of randomly mixed cells (top) and microvascular mesh (bottom) placed on diffusion chamber devices after 2 days of culture in EGM-2 medium. HUVEC:NHDF=9:1, Human CD31 antibody is green to show HUVEC, α-smooth muscle actin (α-SMA) antibody is red to show NHDF, and device is blue. FIG. 3C shows cross-sectional hematoxylin/eosin staining images of retrieved devices after 14 days of subcutaneous implantation. Arrowheads point to blood vessels with erythrocytes inside. FIG. 3D shows density and area percentage of blood vessels at the interface between the device and panniculus carnosus muscle. n=6 in “No cell”, n=6 in “Random”, and n=8 in “Mesh” groups. ** and *** represent P<0.01 and P<0.001, respectively. NS (P>0.05) indicates no significant difference. FIG. 3E shows cross-sectional immunostaining images of human (red) and mouse (green) CD31 antibodies showing the human and mouse blood vessels at the interface between the device and panniculus carnosus muscle. FIG. 2F shows confocal images of perfused lectins bound to human and mouse endothelial cells, confirming the anastomoses between human and mouse vessels. The Fluorescein Ulex Europaeus Agglutinin I (UEA-I) lectins (green) specifically bound to human endothelial cells. The DyLight 594 Labeled Griffonia Simplicifolia lectin I (GSL-I) isolectin B₄ (red) specifically bound to mouse endothelial cells (Kang et al., “Bioengineered Human Vascular Networks Transplanted Into Secondary Mice Reconnect With the Host Vasculature and Re-Establish Perfusion,” Blood 118:6718-6721 (2011), which is hereby incorporated by reference in its entirety). FIG. 3G shows images of blood-perfused human vasculatures anastomosed with mouse vascular system in “Mesh” device after 10 days of subcutaneous implantation. HUVEC-GFP is green and perfused dye DiI in vessels is red. FIG. 3H are representative immunostaining images of mature human vasculatures (human CD31 antibody is green) covered with perivascular cells (α-SMA antibody is red) in retrieved “Random” and “Mesh” device after 10 days of subcutaneous implantation. FIG. 31 shows that the percentage of perivascular cell (PVC) coverage is 19±9% (n=3) and 65±6% (n=6) for “Random” and “Mesh” device, respectively. ** represents P<0.01.

FIGS. 4A-4H demonstrate the improvement of re-vascularization of subcutaneously transplanted rat islets and correction of STZ-induced diabetes in SCID-Beige mice. FIG. 4A is a schematic illustration and a microscopic image of rat islets in a diffusion chamber device wrapped with microvascular meshes (HUVEC:NHDF=9:1). Rat islets expressing GFP are green and microvascular mesh is red. FIG. 4B shows various graphs showing non-fasting blood glucose (BG) concentration of mice after transplantation. Grafts were retrieved after different time points. Most of the mice were kept alive for one week after retrieval while 3 mice from the “Mesh” group were used for perfusion studies prior to retrievals. During 42 days of transplantation (n=6 in “No cell” and “Random”, and n=9 in “Mesh”): * represents P<0.05 and NS (P>0.05) indicates no significant difference. FIG. 4C shows graphs showing BG concentrations during intraperitoneal glucose tolerance tests (IPGTT) after 30 days (top graph) and 90 days (bottom graph) of transplantation. IPGTT at Day 30 (n=3 in all groups): * represents P<0.05 and NS (P>0.05) indicates no significant difference. FIG. 4D shows microscopic images of hematoxylin/eosin staining of rat islets and blood vessels in retrieved devices (Day 42), and the quantified number of blood vessels around a rat islet (within a 200 μm distance). Yellow arrowheads point to blood vessels with erythrocytes inside. White arrows point to rat islets. “No cell” group consists of 11 islets pooled from 3 mice; “Random” group consists of 13 islets pooled from 3 mice; “Mesh” group consists of 12 islets pooled from 3 mice. * and *** represent P<0.05 and P<0.001, respectively. NS (P>0.05) indicates no significant difference. FIG. 4E shows cross-sectional immunostaining images of rat insulin and mouse blood vessels in a retrieved “Mesh” device. Rat insulin antibody is red and mouse CD31 antibody is green. FIG. 4F is an immunostaining image (parallel-section) using human (red) and mouse (green) CD31 antibodies indicate the anastomoses between human and mouse blood vessels. FIG. 4G is a confocal image of perfused blood vessels in re-vascularized rat islets in the “Mesh” group after 42 days of transplantation. FIG. 4H shows fluorescent images of re-vascularized rat islets after 91 days and 112 days of transplantation from the “Mesh” group. The rat islets expressing GFP are green and perfused blood vessels are red.

FIGS. 5A-5F demonstrate that human iPSC-EC-derived microvascular meshes enhance vascularization. FIG. 5A shows fluorescent images of iPSC-EC meshes with different geometries (square, pentagon, hexagon, and octagon). FIG. 5B shows images of iPSC-EC meshes with more complex patterns (“spider web” and “capillary bed”). FIG. 5C shows confocal images of an iPSC-EC mesh at the contracted and junction regions. iPSC-EC expressing GFP is green, human CD31 antibody is red, and nucleus is blue. All samples were imaged after 2 days of culture. FIG. 5D shows hematoxylin/eosin and immunostaining images of retrieved devices after 2 weeks of subcutaneous implantation in SCID-Beige mice. In all in vivo experiments, NHDFs were mixed with iPSC-ECs (iPSC-EC:NHDF=9:1). The yellow arrowheads point to blood vessels with erythrocytes inside. The white dash lines mark the interface between the device and panniculus carnosus muscle. Mouse CD31 antibody is green and α-smooth muscle actin (α-SMA) is red. FIG. 5E are graphs showing the density and area percentage of blood vessels at the interface. n=5 in all groups. *, **, and ** represent P<0.05, P<0.01, and P<0.001, respectively. NS (P>0.05) indicates no significant difference. FIG. 5F shows immunostaining images of human (red) and mouse CD31 (green) antibodies showing the anastomoses between iPSC-EC derived vessels and mouse vessels.

FIGS. 6A-6G show improved diabetes correction by the iPSC-EC microvascular meshes in SCID-Beige mice. FIG. 6A is a microscopic image of rat islets in an iPSC-EC mesh device. The gray color aggregates are rat islets. The white arrow points to iPSC-EC mesh. In all in vivo experiments, NHDFs were mixed with iPSC-ECs (iPSC-EC:NHDF=9:1). FIG. 6B shows graphs illustrating the non-fasting BG concentration of the mice during 91 days of subcutaneous transplantation (n=5 in “No iPSC-EC”, n=6 in “Random iPSC-EC” and “Mesh iPSC-EC”). * represents P<0.05. FIG. 6C are graphs showing BG concentration during intraperitoneal glucose tolerance test (IPGTT) after 30 days and 90 days of transplantation. IPGTT at Day 30 (n=6 in “Normal mice”, “Random iPSC-EC”, and “Mesh iPSC-EC”, and n=4 in “No iPSC-EC”): * represents P<0.05. NS (P>0.05) indicates no significant difference. IPGTT at Day 90 (n=6 in “Normal mice”, n=3 in “No iPSC-EC”, n=4 in “Random iPSC-EC”, and n=6 in “Mesh iPSC-EC”): * represents P<0.05. FIG. 6D is an immunostaining image (parallel-section) of human (red) and mouse CD31 (green) antibodies indicating the anastomoses between human and mouse blood vessels. FIG. 6E shows a confocal image of perfused blood vessels in a re-vascularized rat islet retrieved from the “Mesh” group. FIG. 6F is a hematoxylin/eosin staining image of a retrieved iPSC-EC mesh device. Black arrow points to a rat islet and yellow arrowheads point to blood vessels containing erythrocytes. FIG. 6G shows an immunostaining image of insulin and blood vessels surrounding rat islets in a retrieved iPSC-EC mesh device. Rat insulin antibody is red and mouse CD31 antibody is green.

FIG. 7 is a schematic of a micropillar substrate with a microvascular mesh structure showing various parameters of the micropillar substrate and mesh. The density of mesh opening is equal to the number of micropillars in 1 mm² area.

FIG. 8 shows the organization of HUVECs (expressing GFP) is random in a fibrin matrix on a smooth substrate without micropillars after 2 days of culture.

FIGS. 9A-9C show ASA-enabled HUVEC microvascular meshes with different geometries and dimensions. FIG. 9A shows the arrangement and combination of micropillars with different sizes control organization of HUVECs into square, pentagon, hexagon, and octagon shapes. FIG. 9B illustrates that more complex geometries are realized by changing micropillar arrangement. FIG. 9C shows that micropillar diameters and micropillar-to-micropillar intervals modulate the diameter of fibrin-filled tubular structure, and the size and density of microvascular mesh openings.

FIGS. 10A-10C demonstrates the viability of HUVEC and iPSC-EC cells cultured as microvascular meshes. FIG. 10 shows that a HUVEC (expressing GFP) microvascular mesh maintains square shape for 28 days on a micropillar substrate. FIG. 10B is an image of live (green)/dead (red) staining that indicates a high cell viability in microvascular mesh after 28 days of culture on the micropillar substrate. The quantified cell viability is 97±1% (n=12). FIG. 10C shows that iPSC-EC (expressing GFP) microvascular mesh generates numerous angiogenic sprouts after embedded in a fibrin matrix after 2 weeks of culture.

FIGS. 11A-11B show contraction simulations showing the normal stress distribution in the Y (Cauchy stress component σ₂₂) direction (FIG. 11A) and normal strain distribution E₁₁ and E₂₂ in the X and Y directions (FIG. 11B) on a 4×4 micropillar substrate (dark gray). The initial shape of cells and fibrin matrix is displayed in light gray. The unit of stress is mN/μm². The micropillar diameter is 400 μm and micropillar-to-micropillar distance is 200 μm.

FIG. 12 shows SEM images of a HUVEC hexagon mesh at the inner and boundary regions on a micropillar substrate. The cellular structures wrap around micropillars at the boundary to support entire mesh and prevent its shrinkage. The micropillars are pseudo-colored as blue and HUVECs are pseudo-colored as purple.

FIGS. 13A-13B shows that micropillars assist in the formation of HUVEC microtubules. FIG. 13A shows that HUVECs (expressing GFP) in a fibrin matrix shrink into random clumps in different geometric grooves without micropillars inside. FIG. 13B shows that HUVECs in a fibrin matrix organize into corresponding shapes in different geometric grooves with micropillars inside.

FIGS. 14A-14B show that HUVECs in fibrin matrix gradually shrink into clumps in groove without micropillars inside after 48 hours of culture (FIG. 14A); in contrast, HUVEC microvascular meshes are stable on a micropillar substrate (FIG. 14B).

FIGS. 15A-15B show cross-sectional views of a HUVEC mesh and HUVECs attached to a fibrin matrix. FIG. 15A is a cross-sectional confocal image of a HUVEC mesh (contracted region) organized on a micropillar substrate after 14 days of culture. Human CD31 antibody is green, F-actin is red, and nucleus is blue. FIG. 15B is a cross-sectional confocal image of HUVECs attached around a fibrin matrix. The fibrin matrix is green, human CD31 antibody is red, and nucleus is blue. White arrow points to HUVECs attached on fibrin matrix.

FIGS. 16A-16D illustrate the transfer of microvascular meshes to different substrates. FIG. 16A shows an octagon mesh of HUVECs floating in PBS. FIG. 16B shows pentagon/hexagon meshes of HUVECs transferred to glass cylinders. FIG. 16C shows square/hexagon meshes of HUVECs transferred to PDMS frames. FIG. 16D shows a hexagon mesh of human iPSC-ECs transferred to a rod.

FIGS. 17A-17B show immunostaining images of a HUVEC capillary-like mesh on top of a device. FIG. 17A shows the fluorescent images of stained human CD31 antibody (green), F-actin (red), and nucleus (blue). Nylon grid on the device has auto-fluorescent color. FIG. 17B shows the top-view and cross-sectional view of the HUVEC mesh. Capillary mesh transferred to the device maintained integrity and luminal structure.

FIG. 18 is a schematic illustration of the fabrication process of microvascular mesh device. First, vascular cell suspension in fibrin matrix is placed on a micropillar substrate. Second, microvascular mesh is formed via anchored self-assembly on a micropillar substrate. Third, to transfer microvascular mesh, a diffusion chamber device is placed on top of a microvascular mesh. Fourth, a fibrin matrix is added and another microvascular mesh is placed on the device; After 15 minutes of incubation at 37° C., microvascular meshes are embedded in fibrin matrix and attached to the device. Fifth, two micropillar substrates are removed. Lastly, microvascular meshes and fibrin matrix are trimmed to the size of the device.

FIG. 19 shows immunostaining images of mature blood vessels at the interface between the device and panniculus carnosus muscle after 2 weeks of subcutaneous transplantation. Mouse CD31 antibody is green and a-smooth muscle actin (α-SMA) is red.

FIG. 20 shows images illustrating the gradual formation of blood-perfused human vasculatures which are anastomosed with mouse vascular system during 10 days post transplantation in “Mesh” device. On day 4 and 7, perfused mouse vasculatures are found nearby the microvascular meshes of HUVEC-GFP, however, the blood-perfused human vasculatures have not been generated and connected to mouse vascular system. On day 10, human vasculatures derived from microvascular mesh are functional and connected to mouse vascular system since blood-perfused vessels have overlapped color of green HUVEC-GFP and red dye DiI. It should be noted that original square mesh structures of HUVEC-GFP are gradually re-molded during the vascularization and anastomoses development. In contrast, blood-perfused human vasculatures are not observed in “Random” device under the confocal microscope on Day 10.

FIG. 21 is a schematic illustration of the fabrication process of microvascular mesh device containing islets. The whole process is similar to that with empty devices (FIG. 18) except islets are placed inside the device before microvascular meshes are attached.

FIG. 22 is an image showing transferrable microvascular mesh attached on the device promotes re-vascularization of rat islets transplanted in the poorly vascularized subcutaneous space of SCID-Beige mice for 6 weeks.

FIG. 23 are images of Human iPSC-EC meshes with different geometries transferred to devices. Human iPSC-ECs expressing GFP are green and nylon grids on devices are blue.

FIG. 24A-24C show stacked construct consisting of three alternating microvascular meshes and devices. A high degree of vascularization is observed after two weeks of subcutaneous implantation in SCID-Beige mice. FIG. 24A is a schematic illustration of the fabrication process of stacked construct. Three microvascular meshes and devices are stacked and glued with fibrin matrix, and then subcutaneously transplanted into SCID-Beige mice. FIG. 24B shows digital photos of the construct prior to implantation and after two weeks of implantation, inside the mice right before retrieval. The arrows point to blood vessels on top and side of the device. The original square network of microvascular mesh was not preserved due to the development and re-modeling of vascularization and anastomoses. FIG. 24C shows an immunostaining image of blood vessels (CD31 antibody, green) on the retrieved construct. The construct was distorted during the histological sectioning but abundant vessels were observed on the top of and between devices.

### DETAILED DESCRIPTION

In the following description, reference is made to the accompanying drawings that form a part hereof, and in which is shown by way of illustration specific embodiments which may be practiced. These embodiments are described in detail to enable those skilled in the art to practice the invention, and it is to be understood that other embodiments may be utilized and that structural, logical and electrical changes may be made without departing from the scope of the present invention. The following description of example embodiments is, therefore, not to be taken in a limited sense, and the scope of the present invention is defined by the appended claims.

A first aspect of the present invention relates to a vascularized therapeutic delivery system. This system includes a preparation of cells encapsulated by a biological support material and a microvascular mesh that at least partially surrounds the biological support material encapsulating the preparation of cells. The microvascular mesh includes a network of continuous interconnected tubular structures defined by endothelial cells and an extracellular matrix scaffold.

In accordance with this aspect of the present invention, the “cells” encapsulated by the biological support material include cells in any form, e.g., cell clusters, cell suspensions, cell aggregates, cell monolayers, cells in 3D cell culture, or individually isolated cells. Suitable cells include, without limitation, primary cells, embryonic stem cells, induced pluripotent stem cells, adult stem cells, differentiated embryonic stem cells, differentiated induced pluripotent stem cell cells, differentiated adult stem cells, mesenchymal stem cells, precursor or progenitor cells, genetically modified cells, or cell lines.

The preparation of cells encapsulated by the biological support material, which serve as the therapeutic agent or serve to deliver an expressed or secreted therapeutic agent, may comprise one or more than one cell types. Exemplary suitable cell types include, without limitation, endothelial cells, smooth muscle cells, cardiac muscle cells, cardiac myocytes, epithelial cells, urothelial cells, fibroblasts, myoblasts, chondrocytes, chondroblasts, osteoblasts, keratinocytes, hepatocytes, bile duct cells, pancreatic islet cells, thyroid cells, parathyroid cells, adrenal cells, hypothalamic cells, pituitary cells, ovarian cells, testicular cells, salivary gland cells, adipocytes, embryonic stem cells, adult stem cells, induced pluripotent stem cells, mesenchymal stem cells, neural cells, astrocytes, oligodendrocytes, hematopoietic cells, and any precursor or progenitor cell thereof. Additional exemplary cell types include, without limitation, human umbilical vascular endothelial cells (HUVEC), adult stem cells, blast cells, cloned cells, placental cells, keratinocytes, basal epidermal cells, urinary epithelial cells, salivary gland cells, mucous cells, serous cells, von Ebner's gland cells, mammary gland cells, lacrimal gland cells, eccrine sweat gland cells, apocrine sweat gland cells, MpH gland cells, sebaceous gland cells, Bowman's gland cells, Brunner's gland cells, seminal vesicle cells, prostate gland cells, bulbourethral gland cells, Bartholin's gland cells, Littre gland cells, uterine endometrial cells, goblet cells of the respiratory or digestive tracts, mucous cells of the stomach, zymogenic cells of the gastric gland, oxyntic cells of the gastric gland, insulin-producing P cells, glucagon-producing a cells, somatostatin-producing δcells, pancreatic polypeptide-producing cells, pancreatic ductal cells, Paneth cells of the small intestine, type II pneumocytes of the lung, Clara cells of the lung, anterior pituitary cells, intermediate pituitary cells, posterior pituitary cells, hormone secreting cells of the gut or respiratory tract, gonad cells, juxtaglomerular cells of the kidney, macula densa cells of the kidney, peri polar cells of the kidney, mesangial cells of the kidney, brush border cells of the intestine, striated ducted cells of exocrine glands, gall bladder epithelial cells, brush border cells of the proximal tubule of the kidney, distal tubule cells of the kidney, conciliated cells of the ductulus efferens, epididymal principal cells, epididymal basal cells, hepatocytes, fat cells, type I pneumocytes, pancreatic duct cells, nonstriated duct cells of the sweat gland, nonstriated duct cells of the salivary gland, nonstriated duct cells of the mammary gland, parietal cells of the kidney glomerulus, podocytes of the kidney glomerulus, cells of the thin segment of the loop of Henle, collecting duct cells, duct cells of the seminal vesicle, duct cells of the prostate gland, vascular endothelial cells, synovial cells, serosal cells, squamous cells lining the perilymphatic space of the ear, cells lining the endolymphatic space of the ear, choroid plexus cells, squamous cells of the pia-arachnoid, ciliary epithelial cells of the eye, corneal endothelial cells, ciliated cells having propulsive function, ameloblasts, planum semilunatum cells of the vestibular apparatus of the ear, interdental cells of the organ of Corti, fibroblasts, pericytes of blood capillaries, nucleus pulposus cells of the intervertebral disc, cementoblasts, cementocytes, odontoblasts, odontocytes, chondrocytes, osteocytes, osteoprogenitor cells, hyalocytes of the vitreous body of the eye, stellate cells of the perilymphatic space of the ear, skeletal muscle cells, heart muscle cells, smooth muscle cells, myoepithelial cells, platelets, megakaryocytes, monocytes, connective tissue macrophages, Langerhan's cells, osteoclasts, dendritic cells, microglial cells, neutrophils, eosinophils, basophils, mast cells, plasma cells, helper T cells, suppressor T cells, killer T cells, killer cells, rod cells, cone cells, inner hair cells of the organ of Corti, outer hair cells of the organ of Corti, type I hair cells, cells of the vestibular apparatus of the ear, type II cells of the vestibular apparatus of the ear, type II taste bud cells, olfactory neurons, basal cells of olfactory epithelium, type I carotid body cells, type II carotid body cells, Merkel cells, primary sensory neurons, cholinergic neurons of the autonomic nervous system, adrenergic neurons of the autonomic nervous system, peptidergic neurons of the autonomic nervous system, inner pillar cells of the organ of Corti, outer pillar cells of the organ of Corti, inner phalangeal cells of the organ of Corti, outer phalangeal cells of the organ of Corti, border cells, Hensen cells, supporting cells of the vestibular apparatus, supporting cells of the taste bud, supporting cells of the olfactory epithelium, Schwann cells, satellite cells, enteric glial cells, neurons of the central nervous system, astrocytes of the central nervous system, oligodendrocytes of the central nervous system, anterior lens epithelial cells, lens fiber cells, melanocytes, retinal pigmented epithelial cells, iris pigment epithelial cells, oogonium, oocytes, spermatocytes, spermatogonium, ovarian cells, Sertoli cells, and thymus epithelial cells, or combinations thereof, or cell lines derived therefrom (see, e.g., U.S. Patent Application Publication No. 2013/0203146 A1 to Ying et al., which is hereby incorporated by reference in its entirety).

As noted above, suitable cells include progenitor and/or stem cells. Suitable stem cells may be pluripotent, multipotent, oligopotent, or unipotent cells or cell populations, and include embryonic stem cells, adult stem cells, epiblast cells, primitive ectoderm cells, and primordial germ cells. In another embodiment, suitable stem cells also include induced pluripotent stem (iPS) cells, which are pluripotent stem cells derived from a non-pluripotent cell. See Zhou et al., Cell Stem Cell 4:381-384 (2009); Yu et al., Science 324(5928):797-801 (2009); Yu et al., Science 318(5858):1917-20 (2007); Takahashi et al., Cell 131:861-72 (2007); and Takahashi and Yamanaka, Cell 126:663-76 (2006), which are hereby incorporated by reference in their entirety

In one embodiment of the present invention, the vascularized therapeutic delivery system comprises a preparation of pancreatic islet cells. As described herein, pancreatic islets comprise five distinct cell types: alpha, beta, delta, epsilon, and gamma cells. Alpha cells comprise 15-20% of total islet cells and produce the hormone glucagon, which stimulates the release of glucose from the liver and fatty acids from fat tissue. Beta cells comprise 65-80% of total islet cells and produce insulin, which promotes the uptake and metabolism of glucose by the body's cells, prevents the release of glucose by the liver, causes muscle cells to take up amino acids, and inhibits the breakdown and release of fats. Delta cells comprise 3-10% of total islet cells and produce somatostatin, a strong inhibitor of somatotropin, insulin, and glucagon. Epsilon cells comprise less than 1% of total islet cells and produce ghrelin, which stimulates hunger. Gamma cells comprise 3-5% of total islet cells and secrete pancreatic polypeptide (PP), which regulates endocrine and exocrine pancreatic secretions. The vascularized therapeutic delivery system may comprise a preparation of alpha, beta, delta, epsilon, gamma cells, or any combination thereof.

Suitable cells for encapsulation in the therapeutic delivery system described herein can be derived from any animal capable of generating the desired cells. The animals from which the cells are harvested may be vertebrate or invertebrate, mammalian or non-mammalian, human or non-human. Examples of animal sources include, but are not limited to, primate, rodent, canine, feline, equine, bovine, or porcine. The cells may be obtained from or comprise a primary cell preparation or an immortalized cell preparation. The encapsulated cells may be isolated from the same species as the recipient intending to receive the therapeutic delivery system or from a different species than the intended recipient.

The preparation of cells described herein may comprise cells that have been cultured in vitro, in vivo, and/or ex vivo.

As noted above, in some embodiments, the preparation of cells release, secrete, deliver, or provide one or more therapeutic agents. In some embodiments, the preparation of cells naturally produces the one or more therapeutic agents. In some embodiments, the preparation of cells is a preparation of cells engineered to recombinantly express one or more therapeutic agents.

A therapeutic agent expressed, released, or secreted by the encapsulated preparation of cells of the therapeutic delivery system described herein includes any agent having a desired biological effect, e.g., an agent having a biological activity that modulates, treats and/or cures a disease or condition. The therapeutic agent can be for example, and without limitation, a therapeutic protein, peptide, antibody or fragments thereof, antibody mimetic, or other binding molecule, a nucleic acid molecule, small molecule, hormone, growth factor, angiogenic factor, cytokine, and anti-inflammatory agent.

Protein and/or peptide therapeutic agents which may be produced and released by the vascular therapeutic delivery systems as described herein include, without limitation, peptide hormones such as insulin, glucagon, parathyroid hormone, calcitonin, vasopression, renin, prolactin, growth hormone, the gonadotropins, including chorionic gonadotropin, follicle stimulating hormone, thyroid stimulating hormone, and luteinizing hormone; physiologically active enzymes such as transferases, hydrolases, lyases, isomerases, phosphatases, glycosidases, superoxide dismutase, factor VIII, plasminogen activators; and other therapeutic agents including protein factors such as vascular endothelial growth factor, hepatocyte growth factor, angiopoietin, keratinocyte growth factor epidermal growth factor, insulin-like growth factor, tumour necrosis factor, transforming growth factors, fibroblast growth factors, platelet-derived growth factors, erythropoietin, colony stimulating factors, bone morphogenetic proteins, interleukins, and interferons. Non-protein macromolecules, particularly including polysaccharides, nucleic acid polymers, RNA molecules (e.g., siRNA, shRNA, microRNA), and therapeutic secondary metabolites, including plant products such as vinblastine, vincristine, taxol, and the like may also be delivered using the present system. Small molecular weight compounds may also be delivered.

The therapeutic cell population is encapsulated in a biological support material. As used herein, the term “biological support material” refers to a biological material that supports the growth and survival of living cells under in vitro, in vivo, and/or ex vivo conditions. The biological support material may be fabricated in the form of a gel, sleeve, cuff, sponge, membrane, cube, ring, circle, oval, prism, sphere, tube, sheet, or any other shape useful in biological applications.

The biological support material encapsulating the cells possesses a level of permeability, e.g., semi-permeability, that allows for selective passage of substances into and out of the encapsulated cell preparation. For example, the biological support material exhibits a level of permeability that allows delivery of essential nutrients and oxygen to the encapsulated cells to maintain cell survival and function. The biological support material must allow the passage of therapeutic substances released or secreted from the encapsulated cells to facilitate delivery of such substance(s) to the recipient subject upon implantation of the vascularized delivery system in such recipient subject. At the same time, the biological support material prevents the recipient subject's cells and macromolecules, in particular immune system cells and macromolecules, from passing through and harming the encapsulated cells.

In some embodiments, the biological support material is a synthetic polymer material. Suitable synthetic polymeric materials include, without limitation, polycaprolactone, polylactic acid, polyglycolide, poly(lactic-co-glycolic) acid, polytetrafluoroethylene, nylon, polydimethylsiloxane, polyvinylchloride, polyvinylidene fluoride, polyurethane isocyanates, alginate, cellulose acetate, cellulose nitrate, polysulfone, polyether sulfones, polystyrene, polyurethane, polyvinyl alcohols, polyvinylidenes, polyvinyl chloride copolymers, polyacrylonitrile, poly(acrylonitrile/covinyl chloride), polyamides, polymethylmethacrylate, polyacrylates, polyphosphazenes, polyethylene oxide, and derivatives, copolymers, and mixtures thereof.

In other embodiments, the biological support material is a natural polymer material. Suitable natural polymeric materials include, without limitation, hydrogel, collagen, hyaluronate, fibrin, alginate, agarose, chitosan, bacterial cellulose, elastin, keratin, fibroin, peptide, and combinations thereof.

Other exemplary biological materials suitable for encapsulating the cells, include without limitation, natural gum, agar, agarose, sodium alginate, carrageenan, fucoidan, furcellaran, laminaran, hypnea, eucheuma, gum arabic, gum ghatti, gum karaya, gum tragacanth, locust beam gum, arabinogalactan, pectin, amylopectin, gelatin, hydrophilic colloids such as carboxymethyl cellulose gum or alginate gum cross-linked with a polyol such as propylene glycol, and the like (see, e.g., U.S. Pat. No. 6,632,457 B1 to Sawhney, which is hereby incorporated by reference in its entirety).

In another embodiment, the biological support material is an artificial membrane, e.g., an ultrafiltration membrane or a microfiltration membrane. Those skilled in the art will recognize that ultrafiltration membranes are those having a pore size range of from about 1 to about 100 nanometers, while a microporous membrane has a range of between about 1 to about 10 microns. The manufacture and use of artificial semi-permeable membranes for cell encapsulation and delivery is known in the art (Emerich et al., “Update on Immunoselection Cell Therapy for CNS Diseases,” Cell Transplant 10(1):3-24 (2001), which is hereby incorporated by reference in its entirety).

In some embodiments, part, or all, of the biological support material is partially or completely non-immunogenic. Thus, when the vascularized therapeutic delivery system described herein is implanted into a recipient subject it does not elicit a host response sufficient to result in the rejection of the system or to render it inoperable, for example through degradation.

The second component of the vascularized therapeutic delivery system of the present invention is the microvascular mesh that at least partially surrounds the biological support material encapsulating the preparation of cells. In some embodiments, the microvascular mesh wholly surrounds the encapsulated cells. As described above, the microvascular mesh of the vascularized therapeutic delivery system comprises a network of continuous interconnected tubular structures defined by endothelial cells and an extracellular matrix scaffold.

Endothelial cells are the cells that make up the endothelium which lines the inside surfaces of blood vessels and lymph vessels. The endothelial cells of the microvascular mesh can be any type of endothelial cell, including without limitation, adult vein endothelial cells, adult artery endothelial cells, embryonic stem cell-derived endothelial cells, iPS cell-derived endothelial cells, umbilical vein endothelial cells, umbilical artery endothelial cells, endothelial progenitors cells derived from bone marrow, endothelial progenitors cells derived from cord blood, endothelial progenitors cells derived from peripheral blood, endothelial progenitors cells derived from adipose tissues, endothelial cells derived from adult skin, or a combination thereof. In certain embodiments, the umbilical vein endothelial cells are human umbilical vein endothelial cells (HUVEC). The endothelial cells can be autologous, allogeneic, or xenogeneic with respect to the recipient subject within whom the vascularized therapeutic delivery system will be implanted.

The extracellular matrix scaffold of the microvascular mesh serves to replicate naturally occurring extracellular matrix which is a complex mixtures of structural and non-structural biomolecules. Thus, the extracellular matrix scaffold of the microvascular mesh may comprise any one or more of these biomolecules, including but not limited to, fibronectin, laminin, heparin, collagen, glycosaminoglycan, proteoglycan, elastin, fibrin, fibroin, peptide, and combinations thereof. As described herein, the extracellular matrix scaffold may comprise a single extracellular matrix component such as fibrin.

In some embodiments, the extracellular matrix scaffold may further comprise growth factors, cytokines, and/or antimicrobials that promote, induce, or support endothelial cell organization and vascular development. Suitable growth factors include, e.g., angiogenic growth factors like FGF, bFGF, acid FGF (aFGF), FGF-2, FGF-4, EGF, PDGF, TGF-β, angiopoietin-1, angiopoietin-2, placental growth factor (P1GF), VEGF, PMA (phorbol 12-myristate 13-acetate).

As described supra, the network of continuous interconnected tubular structures of the microvascular mesh are defined by the endothelial cells and extracellular matrix scaffold.

In some embodiments, the tubular structures of the microvascular mesh may be further defined by one or more type of support cell. Suitable support cells include, without limitation fibroblasts, smooth muscle cells, mesenchymal stem cells, and perivascular cells.

Mesenchymal stem cells are adherent, non-hematopoietic cells expressing markers such as CD90, CD105, and CD73, while lacking expression of CD14, CD34, and CD45, and being able to differentiate into adipocytes, chondrocytes, and osteocytes in vitro after treatment with differentiation inducing agents (Ichim et al., “Fibroblasts as a Practical Alternative to Mesenchymal Stem Cells,” J. Transl. Med. 16:212 (2018), which is hereby incorporated by reference in its entirety). Suitable mesenchymal stem cells for inclusion in the microvascular mesh of the present invention include, without limitation, adult human mesenchymal stem cells derived from adipose tissue, peripheral blood, or bone marrow, and neonatal human mesenchymal stem cells derived from the placenta or umbilical cord. Alternatively, the mesenchymal stem cells are derived from induced pluripotent stem cells (Sequiera et al., “Human-Induced Pluripotent Stem Cell-Derived Mesenchymal Stem Cells as an Individual-Specific and Renewable Source of Adult Stem Cells,” Methods Mol. Biol. 1553:183-190 (2017), which is hereby incorporated by reference in its entirety).

Fibroblasts are also a suitable support cell type as they produce extracellular matrix components responsible for maintaining the structural integrity of tissue. Fibroblasts also play an important role in the proliferative phase of wound healing, resulting in deposition of extracellular matrix (Ichim et al., “Fibroblasts as a Practical Alternative to Mesenchymal Stem Cells,” J. Transl. Med. 16:212 (2018), which is hereby incorporated by reference in its entirety). Suitable sources/types of fibroblasts include, without limitation human foreskin fibroblasts, human embryonic fibroblasts, skin fibroblasts cells, vascular fibroblast cells, myofibroblasts, smooth muscle cells, mesenchymal stem cells (MSCs)-derived fibroblast cells, or any combination thereof Alternatively, the fibroblasts are derived from induced pluripotent stem cells (Shamis et al, “iPSC-derived Fibroblasts Demonstrate Augmented Production and Assembly of Extracellular Matrix Proteins,” In Vitro Cell Dev. Biol. Anim. 48(2):112-22 (2012), which is hereby incorporated by reference in its entirety).

Perivascular cells (e.g., pericytes and vascular smooth muscle cells) are also a suitable support cell type given their in vivo role in surrounding the inner endothelial lining, conferring support, and stabilization (see, e.g., Wanj are et al., “Perivascular Cells in Blood Vessel Regeneration,” Biotechnol. J. 8(4):434-447 (2013), which is hereby incorporated by reference in its entirety). During vessel development, both pericytes and vascular smooth muscle cells are recruited to stabilize newly formed vasculature. Perivascular cells wrap around blood vessels and promote vessel maturation by preventing hemorrhaging or leaking of blood vessels. Suitable sources/types of pericytes for inclusion in the microvascular mesh of the present invention include, without limitation, saphenous vein pericytes, cardiac pericytes, umbilical cord pericytes, skeletal muscle pericytes, bone marrow pericytes. Alternatively, the pericytes are derived from induced pluripotent stem cells or mesenchymal stem cells as known in the art (Orlova et al., “Functionality of Endothelial Cells and Pericytes from Human Pluripotent Stem Cells Demonstrated in Cultured Vascular Plexus and Zebrafish Xenografts,” Arteriosclerosis, Thrombosis, & Vascular Biology 34(1): 177-186 (2013), which is hereby incorporated by reference in its entirety.

In some embodiments, the tubular structures of the microvascular mesh have a diameter of about 10 μm to about 300 μm, 10 μm to about 200 μm, 10 μm to about 150 μm, 10 μm to about 100 μm, 10 μm to about 50 μm, 15 μm to about 300 μm, 15 μm to about 200 μm, 15 μm to about 150 μm, 15 μm to about 100 μm, or 15 μm to about 50 μm. In one embodiment, the tubular structures of the microvascular mesh have a diameter of about 15 μm to about 200 μm.

In some embodiments, one or more of the tubular structures are filled with the extracellular matrix scaffold of the microvascular mesh, e.g., fibrin. In another embodiment, one or more of the tubular structures comprise a lumen, i.e., a hollow space or passage through which blood flow.

The network of continuous interconnected tubular structures of the microvascular mesh form a plurality of mesh openings. Using methods described herein in the Examples, the size, shape, and density of the mesh openings can be controlled and tuned to optimize the functionality of the vascular therapeutic delivery system. In one embodiment, the mesh opening size, shape, and density may be uniform throughout the entirety of the microvascular mesh of the inventive therapeutic delivery system. In other embodiment, the size, shape, and/or density of the mesh openings varies (i.e., is not uniform) throughout the microvascular mesh of the inventive therapeutic delivery system.

With respect to mesh opening size, i.e., maximum width of opening, said size can range from about 1 μm to about 1000 μm or greater. In some embodiments, the microvascular mesh has a plurality of openings, where each opening in the mesh ranges in size from about 1 μm to about 1,000 μm, about 1 μm to about 500 μm, about 1 μm to about 467 μm, about 1 μm to about 450 μm, about 1 μm to about 400 μm, about 1 μm to about 350 μm, about 1 μm to about 300 μm, about 1 μm to about 250 μm, about 1 μm to about 200 μm, about 1 μm to about 100 μm, about 1 μm to about 50 μm, about 1 μm to about 10 μm, about 10 μm to about 1,000 μm, about 10 μm to about 500 μm, about 10 μm to about 400 μm, about 10 μm to about 300 μm, about 10 μm to about 200 μm, about 10 μm to about 100 μm, about 20 μm to about 1,000 μm, about 20 μm to about 500 μm, about 20 μm to about 400 μm, about 20 μm to about 300 μm, about 20 μm to about 200 μm, or about 20 μm to about 100 μm.

In some embodiments, the microvascular mesh has a density of openings ranging from 1 to 200 openings/mm² of the mesh, 1 to 100 openings/mm², 1 to 90 openings/mm², 1 to 80 openings/mm², 1 to 70 openings/mm², 1 to 60 openings/mm², 1 to 50 openings/mm², 1 to 40 openings/mm², 1 to 30 openings/mm², 1 to 20 openings/mm², 1 to 10 openings/mm², or 1 to 5 openings/mm². Thus, in some embodiments, the microvascular mesh has a density of openings ranging from 1 to 100 openings/mm².

In some embodiments, the microvascular mesh of the vascularized therapeutic delivery system as described herein has at least 1 to 1,000 openings, 1 to 900 openings, 1 to 800 openings, 1 to 700 openings, 1 to 600 openings, 1 to 500 openings, 1 to 400 openings, 1 to 300 openings, 1 to 200 openings, 1 to 100 openings, 1 to 50 openings, 1 to 25 openings, 1 to 20 openings, 1 to 15 openings, 1 to 10 openings, 1 to 5 openings, 10 to 1,000 openings, 10 to 900 openings, 10 to 800 openings, 10 to 700 openings, 10 to 600 openings, 10 to 500 openings, 10 to 400 openings, 10 to 300 openings, 10 to 200 openings, 10 to 100 openings, 10 to 50 openings, 10 to 25 openings, 10 to 20 openings, 10 to 15 openings, 20 to 100 openings, 30 to 100 openings, 40 to 100 openings, 50 to 100 openings, 60 to 100 openings, 70 to 100 openings, 80 to 100 openings, or 90 to 100 openings.

The mesh openings may possess any two-dimensional geometric shape. In some embodiments, the mesh openings shapes are defined by between 3 to 20 straight sides. For examples, the mesh opening can be a triangle, a quadrilateral (e.g., taking the shape of a kite, rhombus, rhomboid, rectangle, square, trapezoid), a pentagon, a hexagon, a heptagon, an octagon, a nonagon, a decagon, etc. In other embodiments, the mesh opening shape is a star or star-like, having between 5-10 sides. In another embodiment, the mesh openings have one or more circular sides, e.g., circular or oval shapes. In another embodiment the mesh openings have one or more circular sides and one or more straight sides to form a microcapillary geometry. In yet other embodiments, the network of tubular structures and openings of the microvascular mesh are organized in a web pattern, e.g., a spider-web pattern as shown in the Examples herein.

The vascularized therapeutic delivery system described herein may be employed to treat a variety of diseases and conditions requiring a supply of one or more therapeutic agents (e.g., insulin) to a selected subject. The system may contain a homogenous or heterogenous preparation of cells producing one or more therapeutic agents of interest. As described above, the preparation of cells is encapsulated within a biological support material that is permeable or semi-permeable to allow a therapeutic agent (e.g., insulin, glucagon, pancreatic polypeptide, and the like in the case of treating diabetes) secreted by the encapsulated preparation of cells to pass out of the cell delivery system, making the biologically active substance available to target cells outside the vascularized cell delivery system.

Thus, another aspect of the present invention is directed to a method for delivering a therapeutic agent to a subject. This method involves selecting a subject in need of a therapeutic agent; providing the vascularized therapeutic delivery system described herein; and implanting the vascularized therapeutic delivery system into a region of the selected subject suitable for delivering the therapeutic agent.

Suitable therapeutic agents are described herein above. In some embodiments, the therapeutic agent comprises the preparation of encapsulated cells of the vascularized therapeutic delivery system. In other embodiments, the therapeutic agent is released from the preparation of encapsulated cells of the vascularized therapeutic delivery system described herein.

The subject may be a mammalian subject, for example, a human subject. Suitable human subjects include, without limitations, children, adults, and elderly subjects. Alternatively, the subject can be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

In some embodiments, the selected subject has a disease or disorder that can be treated by one or more therapeutic agents described herein. The skilled artisan can readily determine which disorders can be treated using the vascularized therapeutic delivery system described herein. Exemplary disorders include, without limitation, metabolic disorders (e.g., diabetes), cardiovascular disorders, skin disorders, endocrine disorders, neurologic disorders, neurodegenerative disorders, ophthalmic disorders, endothelial cell proliferation or vascularization related disorders, cancer, infectious disorders, inflammatory disorders, immunologic disorders, digestive disorders, vascular disorders, lung disorders, oral disorders, blood disorders, liver disorders, skin disorders, prostate disorders, and kidney disorders.

In some embodiments, the selected subject has a metabolic disorder, where the metabolic disorder is diabetes. For example, the subject may have Type I diabetes (T1D), Type II diabetes (T2D), gestational diabetes, congenital diabetes, cystic fibrosis-related diabetes, hemochromatosis-related diabetes, monogenic diabetes, or drug-induced diabetes (see, e.g., American Diabetes Association, “Diagnosis and Classification of Diabetes Mellitus,” Diabetes Care 34(Suppl 1): S62 (2011), which is hereby incorporated by reference in its entirety).

In some embodiments, where the selected subject has diabetes, the selected subject may be in need of islet cell replacement therapy. Islet cell replacement therapy is a well-established treatment for a subset of patients with diabetes (Bruni et al., “Islet Cell Transplantation for the Treatment of Type 1 Diabetes: Recent Advances and Future Challenges,” Diabetes Metab. Syndr. Obes. 7:211-223 (2014), which is hereby incorporated by reference in its entirety). Thus, in some embodiments, the vascularized cell delivery system comprises a preparation of pancreatic islet cells. Suitable pancreatic islet cells are described above. In some embodiments, the vascularized cell delivery system comprises a preparation of cells engineered to recombinantly express insulin.

In another embodiment, the subject has endocrine disorder, and is in need of an endocrine cell therapy. Thus, the vascularized therapeutic system provided and implanted contains a preparation of endocrine cells that serve as the therapeutic agent or serve to produce and/or release one or more endocrine factors to modulate or treat the endocrine disorder of the subject. In another embodiment, the subject has a neurologic or neurodegenerative disorder and is in need of neurological cell therapy. Thus, the vascularized therapeutic system provided and implanted contains a preparation of neurons, astrocytes, oligodendrocytes or other neurological cells that serve as the therapeutic agent or serve to produce and/or release therapeutic agents capable of modulating and/or treating the neurological disorder. In another embodiment, the subject has an ophthalmic disorder and is need of an ophthalmic cell therapy. Thus, the vascularized therapeutic system provided and implanted contains a preparation of ophthalmic cells that serve as the therapeutic agent or serve to produce and/or release one or more therapeutic agents capable of modulating and/or treating the ophthalmic disorder. In another embodiment, the subject has a liver disorder and is in need of hepatocyte therapy. Thus, the vascularized therapeutic system provided and implanted contains a preparation of hepatocytes or related support cells that serve as the therapeutic agent or serve to produce and/or release therapeutic agents capable of modulating and/or treating the liver disorder. In another embodiment, the subject has a cardiac disorder, and is need of cardiac cell therapy. Thus, the vascularized therapeutic system provided and implanted contains a preparation of cardiac cells that serve as the therapeutic agent or serve to produce and/or release therapeutic agents capable of modulating and/or treating the cardiac disorder. In another embodiment, the subject has a muscular disorder, and is need of muscle cell therapy. Thus, the vascularized therapeutic system provided and implanted contains a preparation of muscle cells that serve as the therapeutic agent or serve to produce and/or release therapeutic agents capable of modulating and/or treating the muscular disorder. In another embodiment, the subject has a gastrointestinal disorder, and is need of gastrointestinal cell therapy. Thus, the vascularized therapeutic system provided and implanted contains a preparation of gastrointestinal cells (e.g., intestinal, stomach, bile, pancreatic, liver cells) that serve as the therapeutic agent or serve to produce and/or release therapeutic agents capable of modulating and/or treating the gastrointestinal disorder. In another embodiment, the subject has a renal disorder, and is need of renal cell therapy. Thus, the vascularized therapeutic system provided and implanted contains a preparation of renal cells that serve as the therapeutic agent or serve to produce and/or release therapeutic agents capable of modulating and/or treating the renal disorder. In another embodiment, the subject has a wound, chronic or acute, and is need of wound therapy. Thus, the vascularized therapeutic system provided and implanted contains a preparation of cells involved in wound healing that serve as the therapeutic agent or serve to produce and/or release therapeutic agents capable of providing factors required for wound healing.

In some embodiments, when the preparation of cells encapsulated by the biological support material within the vascularized cell delivery system described herein are protected from the host immune system, allowing the preparation of cells to be derived from any suitable source, i.e., human or non-human. Examples of suitable non-human sources include, without limitation, non-human primates, pigs, bovids, equids, felids, canids, and rodents. Thus, as described above, the preparation of cells may be autologous, allologous, or xenologous to the selected subject. In some embodiments, the preparation of cells are stem or progenitor cells, including induced pluripotent stem cells that differentiate into mature cell capable of delivering a therapeutic agent.

In some embodiments, the microvascular mesh of the vascularized therapeutic delivery system comprises endothelial cells obtained from the selected subject. In accordance with this embodiment, the endothelial cells are autologous. For example, the endothelial cells may be autologous HUVEC cells or autologous induced pluripotent stem cells which have been differentiated into endothelial cells. In other embodiments, the endothelial cells may be allogeneic or xenogeneic with respect to a selected subject within whom they may be implanted.

In some embodiments, the vascularized therapeutic delivery system is implanted using minimally invasive surgical techniques such as laparoscopy. The vascularized therapeutic delivery system may be implanted percutaneously, subcutaneously, intradermally, intraperitoneally, intrathoracically, intramuscularly, intraarticularly, intraocularly, or intracerebrally depending on the therapeutic agent being delivered, condition to be treated, and tissue or organ targeted for delivery.

The vascularized therapeutic delivery system may be implanted into, adjacent to, or near the abdominal cavity, thoracic cavity, eye, spleen, ear, heart, colon, liver, kidney, breast, joint, ovary, testicle, bone marrow, or skin of a selected subject. In some embodiments, the vascularized therapeutic delivery system is implanted into a subcutaneous region or space of the selected subject.

Prior art cell replacement therapies often fail as a result of inadequate diffusion of nutrients and oxygen to the implanted cells. The vascularized therapeutic delivery system described herein overcomes this deficiency by enabling the formation of an interconnected network of capillaries and/or blood vessels between the host recipient subject and the implanted vascularized cell delivery system. This revascularization process may initiate as soon as 1, 2, 3, 4, 5, 6, 7, 8, 9, 10 or >10 days post-implanting of the vascularized cell delivery system into a region of the selected subject in need of cell based therapy. Thus, the vascularized cell delivery system described herein allows nutrients present in the selected subject to pass through the biological support material to provide essential nutrients to the encapsulated preparation of cells. At the same time, such a biological support material prevents the recipient subject's cells, more particularly, their immune system cells and inflammatory factors, from passing through the biological support material to harm the preparation of cells encapsulated in the system. For example, in the case of diabetes, this approach may allow glucose and oxygen (e.g., contained within the body) to stimulate β cells of the implanted vascularized cell delivery system to release insulin as required by the body in real time while preventing host immune system cells from recognizing and destroying the implanted cells.

In some embodiments, the vascularized cell delivery system is monitored regularly (e.g., multiple times a day, daily, weekly, twice weekly, monthly, bi-monthly, annually, semi-annually, or any amount of time there between). Regular monitoring may ensure that the preparation of cells encapsulated by the vascularized cell delivery system are functioning adequately.

To facilitate monitoring, the vascular therapeutic system described herein may comprise one or more contrast agents (e.g., in the microvascular mesh) to facilitate in vivo monitoring of system placement, location of system at some time point after implantation, health of the system, deleterious effects on non-target cell types, inflammation, and/or fibrosis. Suitable contrast agents include, without limitation, nanoparticles, nanocrystals, gadolinium, iron oxide, iron platinum, manganese, iodine, barium, microbubbles, fluorescent dyes, and others known to those of skill in the art.

Methods of in vivo monitoring include but are not limited to confocal microscopy, 2-photon microscopy, high frequency ultrasound, optical coherence tomography (OCT), photoacoustic tomography (PAT), computed tomography (CT), magnetic resonance imaging (MRI), single photon emission computed tomography (SPECT), and positron emission tomography (PET). These alone or combined can provide useful means to monitoring the implantable vascular therapeutic delivery system.

The methods described herein may further involve retrieving and replacing the vascularized therapeutic delivery system in the selected subject. For example, retrieval of the vascularized cell delivery system described herein may be desirable when the preparation of cells encapsulated by the biological support material of the vascularized cell delivery system described herein ceases to release adequate amounts of a biologically active agent (e.g., insulin). Following retrieval, the retrieved vascularized cell delivery system may be replaced by another vascularized cell delivery system to continue providing therapeutic delivery to the selected subject. A second or subsequently implanted vascularized therapeutic delivery system may be implanted in the same or a different location.

A further aspect of the present invention is directed to a method of producing a vascularized therapeutic delivery system. This method involves providing a monolithic substrate comprising a planar surface interrupted by a plurality of micropillars. This method further involves applying endothelial cells suspended in an extracellular matrix material to the planar surface of the monolithic substrate, and culturing the suspended endothelial cells under conditions effective for the endothelial cells to organize into a microvascular mesh, where the microvascular mesh comprises a network of continuous interconnected tubular structures defined by the endothelial cells and extracellular matrix material. The microvascular mesh is transferred to an outer surface of an implantable cell delivery construct comprising a preparation of cells to produce the vascularized therapeutic delivery system. An exemplary process for carrying out this aspect of the present invention is described in the Examples herein.

The monolithic substrate is comprised of any material conducive to culturing cells. Thus, in some embodiments, the monolithic substrate comprises a polymer material such as a polysiloxane, polystyrene, polyvinyl chloride, silica, a carbon-based polymer, polydimethylsiloxane (PDMS), a polyacrylamide, a polyacrylate, a polymethacrylate, or mixtures thereof. The surface of the substrate can optionally be coated with one or more bioreagents suitable for minimizing cell adhesion to the surface. Suitable bioreagents are known in the art and include, without limitation non-ionic surfactants such as Pluronic® F127.

As described herein, the monolithic substrate comprises a planar surface interrupted by a plurality of micropillars. The plurality of micropillars project vertically from the surface of the substrate, with each of the plurality of micropillars having a proximal end attached to the surface of the substrate and a distal end that projects away from the surface of the substrate. In some embodiments, the micropillars have a geometry that maintains the same diameter from the proximal end attached to the surface of the substrate to the distal end that projects away from the surface of the substrate. In some embodiments, the micropillars have a geometry that tapers in diameter from the proximal end attached to the surface of the substrate to the distal end that projects away from the surface of the substrate. In some embodiments, the plurality of micropillars are uniform in size and/or geometry across the substrate, in other embodiments the plurality of micropillars are not uniform in size and/or geometry across the substrate.

The plurality of micropillars can be of any three-dimensional shape, for example and with out limitation, a cylinder, sphere, torus, cube, cone, rectangular prism, triangular prism, pentagonal prism, hexagonal prism, heptagonal prism, octagonal prism, nonagonal prism, decagonal prism, etc. In some embodiments, the plurality of micropillars on the substrate all have the same shape. In other embodiments, the plurality of micropillars on the substrate have a combination of different shapes.

In some embodiments, each of the plurality of micropillars has a height ranging from about 10 μm to about 1000 μm, about 10 μm to about 900 μm, about 10 μm to about 800 μm, about 10 μm to about 700 μm, about 10 μm to about 600 μm, about 10 μm to about 500 μm, about 10 μm to about 400 μm, about 10 μm to about 300 μm, about 10 μm to about 200 μm, about 10 μm to about 100 μm, about 10 μm to about 50 μm, about 20 μm to about 1000 μm, about 20 μm to about 900 μm, about 20 μm to about 800 μm, about 20 μm to about 700 μm, about 20 μm to about 600 μm, about 20 μm to about 500 μm, about 20 μm to about 400 μm, about 20 μm to about 300 μm, about 20 μm to about 200 μm, about 20 μm to about 100 μm, or about 20 μm to about 50 μm. In some embodiments, each of the plurality of micropillars has a height of about 100 μm to about 400 μm. The height of the micropillars on the surface of the substrate may be uniform. Alternatively, the height of the micropillars on the surface of the substrate may vary.

In some embodiments, each of the plurality of micropillars has a width ranging from about 10 μm to about 1000 μm, about 10 μm to about 900 μm, about 10 μm to about 800 μm, about 10 μm to about 700 μm, about 10 μm to about 600 μm, about 10 μm to about 500 μm, about 10 μm to about 400 μm, about 10 μm to about 300 μm, about 10 μm to about 200 μm, about 10 μm to about 100 μm, about 10 μm to about 50 μm, about 20 μm to about 1000 μm, about 20 μm to about 900 μm, about 20 μm to about 800 μm, about 20 μm to about 700 μm, about 20 μm to about 600 μm, about 20 μm to about 500 μm, about 20 μm to about 400 μm, about 20 μm to about 300 μm, about 20 μm to about 200 μm, about 20 μm to about 100 μm, or about 20 μm to about 50 μm. In some embodiments, each of the plurality of micropillars has a width of about 100 μm to about 400 μm. The width of the micropillars on the surface of the substrate may be uniform. Alternatively, the width of the micropillars on the surface of the substrate may vary.

The distance between adjacent micropillars on the surface of the substrate may be uniform or varied. In some embodiments, the distance between adjacent micropillars on the surface of the substrate ranges from about 10 μm to about 500 μm, about 10 μm to about 400 μm, about 10 μm to about 300 μm, about 10 μm to about 200 μm, about 10 μm to about 100 μm, about 10 μm to about 50 μm, about 50 μm to about 500 μm, about 50 μm to about 400 μm, about 50 μm to about 300 μm, about 50 μm to about 200 μm, or about 50 μm to about 100 μm.

In some embodiments, the distance between adjacent micropillars on the surface of the substrate is about 50 μm to about 500 μm.

In some embodiments, the micropillars are sized and spatially arranged on the surface of the planar substrate in a manner effective to define a mesh opening geometry. For example, the micropillars may be sized and spatially arranged in a manner to control and tune the size and shape of the mesh openings. As described supra, the mesh openings can have a triangular, quadrilateral, pentagon, hexagon, heptagon, octagon, nonagon, decagon, circular, or microcapillary geometry. In some embodiments, the network of tubular structures and openings of the microvascular mesh are organized in a web pattern, e.g., a spiral or spiderweb pattern. As described above, the microvascular mesh openings range in size from 20 μm to 1,000 μm.

The number of micropillars on the surface of the substrate controls the density of mesh openings in the microvascular mesh construct. In some embodiments, the substrate has a density of micropillars on its surface ranging from 1 to 200 micropillars/mm², 1 to 100 micropillars/mm², 1 to 90 micropillars/mm², 1 to 80 micropillars/mm², 1 to 70 micropillars/mm², 1 to 60 micropillars/mm², 1 to 50 micropillars/mm², 1 to 40 micropillars/mm², 1 to 30 micropillars/mm², 1 to 20 micropillars/mm², 1 to 10 micropillars/mm², or 1 to 5 micropillars/mm².

In some embodiments, the surface of the substrate as described herein has at least 1 to 1,000 micropillars, 1 to 900 micropillars, 1 to 800 micropillars, 1 to 700 micropillars, 1 to 600 micropillars, 1 to 500 micropillars, 1 to 400 micropillars, 1 to 300 micropillars, 1 to 200 micropillars, 1 to 100 micropillars, 1 to 50 micropillars, 1 to 25 micropillars, 1 to 20 micropillars, 1 to 15 micropillars, 1 to 10 micropillars, 1 to 5 micropillars, 10 to 1,000 micropillars, 10 to 900 micropillars, 10 to 800 micropillars, 10 to 700 micropillars, 10 to 600 micropillars, 10 to 500 micropillars, 10 to 400 micropillars, 10 to 300 micropillars, 10 to 200 micropillars, 10 to 100 micropillars, 10 to 50 micropillars, 10 to 25 micropillars, 10 to 20 micropillars, 10 to 15 micropillars, 20 to 100 micropillars, 30 to 100 micropillars, 40 to 100 micropillars, 50 to 100 micropillars, 60 to 100 micropillars, 70 to 100 micropillars, 80 to 100 micropillars, or 90 to 100 micropillars.

The micropillar containing substrate can be of any desired shape and size to control the shape and size of the microvascular mesh. Alternatively, the microvascular mesh can be trimmed to the desired shape and size to fit the encapsulated cell preparation as desired. The micropillar containing substrate can range from 0.1 to 10 cm in length by 0.1 to 10 cm in width.

Suitable types and sources of endothelial cells and extracellular matrix material (i.e., fibronectin, laminin, heparin, collagen, glycosaminoglycan, proteoglycan, elastin, fibrin, fibroin, and combinations thereof) to apply to the micropillar containing surface of the substrate are described supra. In other embodiments one or more types of support cells, i.e., fibroblasts, mesenchymal stem cells, perivascular cells as described supra, are also suspended in the extracellular matrix for applying to the substrate. The density of cells suspended in the extracellular material and applied to the surface of the substrate depends on the size and dimensions of the substrate. Generally a density of approximately 1×10⁴ or 1×10⁵ cells/ml to about 1×10¹⁰ cells/mL or more is suitable for distribution across the surface of the substrate.

The suspension of cells in extracellular matrix material may further comprise one or more additional cell specific growth and/or differentiation factors to enhance cell growth, differentiation, survival, and organization into the network of tubular structures. These factors include supplements (e.g., glutamine, non-essential amino acids), growth factors (e.g., epidermal growth factors, fibroblast growth factors, transforming growth factor/bone morphogenetic proteins, platelet derived growth factors, insulin growth factors, cytokines), extracellular matrix proteins (e.g., fibronectin, laminin, heparin, collagen, glycosaminoglycan, proteoglycan, elastin, chitin derivatives, fibrin, and fibrinogen), angiogenic factors (e.g., FGF, bFGF, acid FGF (aFGF), FGF-2, FGF-4, EGF, PDGF, TGF-beta, angiopoietin-1, angiopoietin-2, placental growth factor (P1GF), VEGF, and PMA (phorbol 12-myristate 13-acetate)), and signaling factors and/or transcription factors.

After applying the endothelial cells suspended in the extracellular matrix material to the surface of the substrate, the cell suspension is cultured under conditions effective for the endothelial cells and support cells, if present in the suspension, to organize into a network of continuous interconnected tubular structures. These conditions involve incubation under temperatures suitable to polymerize or gelatinize the extracellular matrix material (e.g., about 30-40° C.) and culture in the presence of cell culture media containing essential nutrients and growth factors to facilitate cell survival, growth, differentiation, and organization. The optimal media varies depending on the cellular composition, i.e., endothelial cells alone or in combination with one or more types of support cells, and can be readily determined by one of skill in the art. Organization of the endothelial cells and support cells (if present) into the network of continuous interconnected tubular structures occurs overnight and over the course of one to five or more days in culture. The characteristics of the microvascular mesh created in accordance with this method, e.g., mesh opening size, geometry, density as well as tubular structure and diameter, are described in detail supra.

The microvascular mesh described in accordance with the methods described above is transferred to an outer surface of an implantable cell delivery construct comprising a preparation of cells. Suitable preparations of cells in the implantable cell delivery construct are described supra and include, without limitation one or more of smooth muscle cells, endothelial cells, cardiac muscle cells, cardiac myocytes, epithelial cells, urothelial cells, fibroblasts, myoblasts, chondrocytes, chondroblasts, osteoblasts, keratinocytes, hepatocytes, bile duct cells, pancreatic islet cells, thyroid cells, parathyroid cells, adrenal cells, hypothalamic cells, pituitary cells, ovarian cells, testicular cells, salivary gland cells, adipocytes, embryonic stem cells, induced pluripotent stem cells, mesenchymal stem cells, neural cells, astrocytes, oligodendrocytes, hematopoietic cells, any precursor or progenitor cell thereof, and any of the aforementioned cells engineered to express and/or secrete a therapeutic agent, such as a therapeutic protein, hormone, growth factor, cytokine, etc.

To remove the microvascular mesh from the micropillar substrate the substrate can be immersed into a phosphate buffered solution which facilitates the release of the microvascular mesh. The mesh stays afloat in the solution and can be picked up and transferred to the surface of an implantable cell delivery construct using a rod, tweezers, pipet, spatula, or similar tool. Alternatively, the surface of the implantable cell delivery system is coated in an extracellular matrix material, e.g., fibronectin, fibrin, collagen, etc., and placed on top of the micropillar substrate. The microvascular mesh will adhere to the implantable cell delivery system and the micropillar substrate can be peeled away to facilitate the transfer of the mesh to the implantable cell delivery device.

In accordance with this aspect of the present invention, one or more of the vascular therapeutic delivery systems may be stacked together vertically to scale up the size of the system to ensure sufficient therapeutic agent delivery. The stacked devices can be held together with an extracellular matrix material layer between each device and/or by encapsulation in an angiogenic support material. Suitable angiogenic support materials include, without limitation, hydrogel, collagen, hyaluronate, fibrin, alginate, agarose, chitosan, bacterial cellulose, elastin, keratin, fibroin, and combinations thereof.

In some embodiments, the methods of producing a vascularized therapeutic delivery system further involves selecting a subject in need of a therapeutic agent and implanting the vascularized therapeutic delivery system into said subject. Suitable subjects and methods of implanting are described supra.

Another aspect of the present invention is directed to the microvascular mesh as described herein and made in accordance with the methods described herein. Another aspect of the present invention is directed to the microvascular mesh on the micropillar substrate as described herein. Production of the microvascular mesh on the micropillar substrate imparts elasticity and resilience to the microvascular mesh. As a result the mesh is resistant to shrinking, and in some embodiments, it can retain its shape, even when removed from the substrate comprising the plurality of micropillars.

Although the present invention has been described for the purpose of illustration, it is understood that such detail is solely for that purpose and variations can be made by those skilled in the art without departing from the spirit and scope of the invention which is defined by the following claims.

### EXAMPLES

The examples below are intended to exemplify the practice of embodiments of the disclosure but are by no means intended to limit the scope thereof.

### Materials and Methods For Examples 1-5:

### Fabrication of Micropillar Substrate

The micropillar substrate was composed of polydimethylsiloxane (PDMS) and fabricated using standard soft lithography at the Cornell NanoScale Facility (CNF). Briefly, a photomask was prepared using a mask writer (DWL2000, Heidelberg Instruments). The silicon wafer was spin-coated with SU-8 100 photoresist (MicroChem) at 500 rpm for 30 sec and then 1300 rpm for 30 sec. The coated wafer was covered with the photomask and exposed to UV light in a UV photolithography machine (ABM Contact Aligner) for 55 seconds. After being developed and post-baked, the SU-8 master wafer was fabricated. The master wafer contained 200 μm-deep microwells with various geometries. The master wafer was used to create PDMS (Sylgard 184, Dow Corning) micropillars. A mixture (10:1, w:w) of Sylgard 184 silicone elastomer components was casted onto the master wafer, cured at 60° C. overnight, and peeled off from the master to obtain a PDMS micropillar substrate.

### Cell Culture

Normal human umbilical vein endothelial cells (HUVECs, passage 4 to 6, Lonza) or HUVECs expressing GFP were cultured in EGM-2 medium (Lonza). Normal human dermal fibroblasts (NHDF, passage 4 to 10, Lonza) were cultured in FGF-2 medium (Lonza). Cells were plated at a density of ˜10,000 cells/cm² and grown at 37° C. and 5% CO₂ incubator to ˜80% confluence over ˜6 days prior to experiment. Cells were not tested for mycoplasma contamination in house but Lonza certified them free of contamination.

The human iPSC-ECs were generated as previously described (James et al., “Expansion and Maintenance of Human Embryonic Stem Cell-Derived Endothelial Cells by TGFβ Inhibition is Id1 Dependent,” Nat. Biotechnol. 28:161-166 (2010), which is hereby incorporated by reference in its entirety). Briefly, embryonic bodies were generated from iPSC (LN4, a subclone of iPS.C2a which was initially reprogrammed from human foreskin fibroblasts (Song et al., “Engraftment of Human Induced Pluripotent Stem Cell-Derived Hepatocytes in Immunocompetent Mice via 3D Co-Aggregation and Encapsulation,” Sci. Rep. 5:16884 (2015), which is hereby incorporated by reference in its entirety)) and cultured on non-adherent plates in advanced DMEM/F12 medium (Gibco) supplemented with 20% (v/v) knockout serum replacement (Invitrogen), non-essential amino acids (Gibco), L-glutamine (Invitrogen), penicillin/streptomycin (Invitrogen), β-mercaptoethanol (Gibco), and then cytokines were added sequentially: 20 ng/mL bone morphogenetic protein-4 (BMP-4, R&D Systems) (Days 0-7), 10 ng/mL Activin A (R&D Systems) (Days 1-4), and 8 ng/mL fibroblast growth factor-2 (FGF-2, Peprotech) (Days 2-14). On day 4, embryonic bodies were transferred to adherent conditions on Matrigel (BD Biosciences) coated plates and medium was supplemented with 25 ng/mL vascular endothelial growth factor-A (VEGF-A, Peprotech) (Days 4-14), 10 μM SB431542 (Tocris) (Day 7-remainder of experiment). After 14 days cell mixtures were dissociated using Accutase (eBioscience) and sorted for CD31⁺ endothelial cells. Purified iPSC-ECs were cultured in EGM-2 medium for further experiment.

### Anchored Self-Assembly of Endothelial Cells on Micropillar Substrate to Form a Microvascular Mesh

PDMS micropillar substrates were autoclaved, treated with UV ozone cleaner (Model 18, Jelight) for 10 minutes, placed in a 24-well plate, and coated with 1% (w/v) Pluronic® F127 (Sigma) solution before cell seeding to prevent cell attachment on PDMS surface and to facilitate cell assembly.

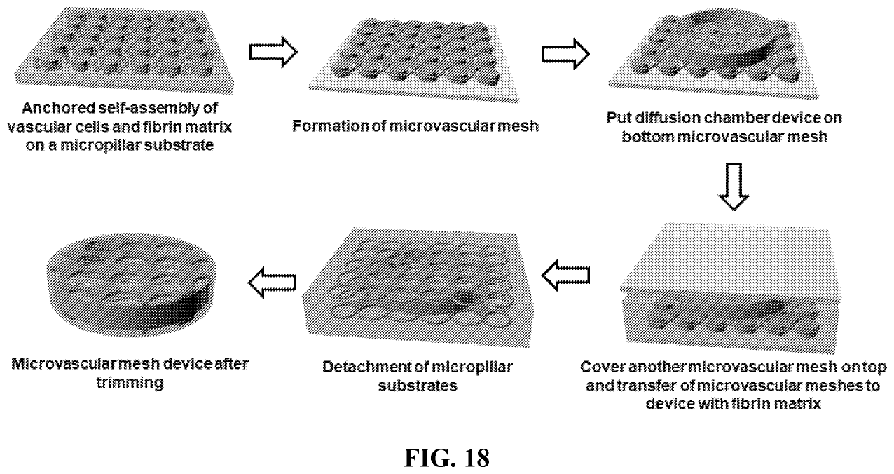

To form microvascular meshes (FIG. 18), cells were suspended in fibrin solution (3 mg/mL fibrinogen (from bovine plasma, Sigma) and 1.0 U/mL thrombin (from bovine plasma, Sigma)) at a concentration of 8.0×10⁶ cells/mL and poured over a PDMS micropillar substrate. Excess cell suspension was scraped off with a cover glass. The cells in fibrin solution homogeneously filled the interspaces between micropillars. After 15 minutes of incubation at 37° C., a fibrin gel was formed and cells embedded inside were cultured in EGM-2 medium. Microvascular meshes formed between micropillars after overnight culture and further stabilized during subsequent 2 days of culture. All the in vitro characterization of microvascular mesh was performed after 2 days of culture. In some experiments, fibrinogen conjugated with Alexa Fluor™ 488 (Molecular Probes) was mixed with normal fibrinogen to form fluorescent fibrin gel (1:200 dilution).

### Simulation

A free energy that has a passive elastic contribution from the fibrin matrix and an active contractile contribution from the cells was postulated, and whether they act in parallel was considered. The effects of contractility are assumed to be greater than the poroelastic effects in the contracting microtissue, thus the effect of pore pressure is neglected, essentially considering the fibrin matrix as a Neo-Hookean compressible foam on which the cells are acting. Both of these assumptions are prevalent in the literature for modeling and simulation of contractile microtissue (Wang et al., “Necking and Failure of Constrained 3D Microtissues Induced by Cellular Tension,” Proc. Natl. Acad. Sci. U.S.A. 110:20923-20928 (2013) and Ahmadzadeh et al., “Modeling the Two-Way Feedback Between Contractility and Matrix Realignment Reveals a Nonlinear Mode of Cancer Cell Invasion,” Proc. Natl. Acad. Sci. U.S.A. 114:E1617-E1626 (2017), each of which is hereby incorporated by reference in its entirety), adequate for capturing the key features of the response of microtissues. The assumed free energy has the form:

$$U ( {I}_{C} , J , {η}_{iso} ) = {U}_{p} ( {I}_{C} , J ) + {U}_{a} ( J , η ) = \frac{μ}{2} ( {I}_{C} - 3 - 2 ln ( J ) ) + \frac{λ}{2} {( ln ( J ) )}^{2} + {η}_{iso} {β ( ln ( J ) )}^{2}$$

In the last part of the equation, the first two terms correspond to the compressible Neo-Hookean formulation for the homogenized fibrin network, and the last term to the homogenized active contractile action of the cells. Decoupling the energetic contributions is a prevalent assumption in the field of computational models (Ahmadzadeh, H. et al., “Modeling the Two-Way Feedback Between Contractility and Matrix Realignment Reveals a Nonlinear Mode of Cancer Cell Invasion,” Proc. Natl. Acad. Sci. U.S.A. 114:E1617-E1626 (2017), which is hereby incorporated be reference in its entirety). Considering both the active (cell) and passive (fibrin) parts of the free energy is critical in the prediction of the aggregate response of the composite microtissue. Where I_{C} is the first principal invariant of the right Cauchy-Green deformation gradient C_{IJ}=F_{iI}F_{jJ}, J is the third principal invariant of the deformation gradient F_{iJ}=∂xᵢ/∂X_{J} where small letters refer to the reference and capital refer to current configuration. Where I_{C} is the first principal invariant of the right Cauchy-Green deformation gradient, J is the third principal invariant of the deformation gradient. The shear modulus of matrix is denoted as μ and Lame's first parameter is μ. The cell isotropic contractile activation ηᵢₛₒ is a dimensionless parameter that ranges from 0 to 1 characterizing the intensity of the cellular contraction. The chemo-mechanical stiffness modulus β has units of stress. This free energy form is introduced to the FEniCS platform (Logg et al., “Automated Solution of Differential Equations by the Finite Element Method: The FEniCS Book,” Edn. 1. (Springer-Verlag Berlin Heidelberg, 2012), which is hereby incorporated by reference in its entirety) to conduct the finite element simulations in order to be able to predict and optimize the post-contraction mechanical characteristics of the system. The analysis is nonlinear, as material and geometric nonlinearities are considered and also the contact formulation is nonlinear. Linear triangular elements are employed for the simulation in FEniCS.

On a 4×4 micropillar substrate, the micropillars are considered rigid and frictionless contact is considered between the cell/fibrin composite and the micropillars. Loading is induced through ramping of the isotropic activation to its maximum value, to mimic the effect of cell contraction on the fibrin network. The micropillar diameter is 400 μm and pillar-to-pillar distance is 200 μm. Young's modulus of the matrix is taken to be if E=1.9 mN/μm ² interpolating from the results of Ghajar et al., “Mesenchymal Stem Cells Enhance Angiogenesis in Mechanically Viable Prevascularized Tissues Via Early Matrix Metalloproteinase Upregulation,” Tissue Eng. 12:2875-2888 (2006), which is hereby incorporated by reference in its entirety). Poisson's ratio is set at ν=0.3, and the chemo-mechanical stiffness modulus β=2 mN/μm² was calibrated to the experimental results to match the observed in-plane displacements. The shear modulus of matrix is obtained through

$$λ = \frac{Ev}{( 1 + v ) ( 1 - 2 v )} .$$

and Lame's first parameter through

$$μ = \frac{E}{2 ( 1 + v )}$$

The simulations are under plane-stress conditions, consistent with the calibration procedure to obtain the model parameters. A simulation is performed starting from zero activation, ramping up to full activation to study the effect of cell contraction, leading to large deformations to the microtissue as observed in the simulations and in agreement with experimental observations. The units of stress in the results are mN/μm² and displacement units are μm. The strains for this large deformation problem are quantified through the Euler-Lagrange strain tensor defined as E_{IJ}=F_{iI}F_{jJ}−I_{IJ} where I is the unit second order tensor.

### Transfer of Microvascular Meshes

Due to intrinsic elasticity and resilience, microvascular mesh on micropillar substrate can be manipulated and transferred to different substrates. An intact microvascular mesh at centimeter scale could float after immersing the micropillar substrate in phosphate buffered saline solution. When a rod was placed beneath the floating microvascular mesh and gently lifted up, the microvascular mesh wraps onto the rod.

Alternatively, to transfer a microvascular mesh onto different substrates, a PDMS frame (pre-soaked in 50 μg/mL fibronectin solution for 1 hour at 37° C. to promote cell adhesion) or glass cylinder was placed on top of the micropillar substrate. During overnight culture, while the cells between micropillars organized into a mesh, the PDMS frame or glass cylinder provided cell adhesive points over the entire mesh. The microvascular mesh can then be easily transferred by gently peeling the frame/cylinder off of the micropillar substrate.

### Vascularization of Diffusion Chamber Devices by Microvascular Meshes in Mouse Subcutaneous Space

Circular PDMS frames (inner diameter: 5 mm, outer diameter: 6 mm, thickness: 1 mm) were cut from a PDMS membrane using biopsy punches (diameters: 5 and 6 mm, Sklar). Diffusion chamber devices without islets were fabricated by attaching a circular nylon grid (diameter: 6 mm, pore size: 70 μm, Component Supply) to either side of the PDMS frame. PDMS pre-polymer solution was used as glue. After being coated with 1% (w/v) Pluronic® F127 (Sigma) solution overnight, the void space in chamber device was filled with fibrin matrix (18 mg/mL) to minimize the cell infiltration and tissue ingrowth into the chamber in vivo.

In all in vivo experiments, microvascular mesh was made of ECs and NHDFs (ECs:NHDFs=9:1). NHDFs were added to enhance vascularization and maturation of vasculatures. As previously described, microvascular mesh was assembled in the same way on the micropillar substrate. The parameters of a micropillar substrate were as follows: substrate size was 1×1 cm, micropillar diameter and height were 400 μm and 200 μm, and micropillar-to-micropillar interval was 200 μm. The volume of the interspace between micropillars was approximately 13.6 μL. The total cell number on the micropillar substrate was approximately 1.1×10⁵. After microvascular mesh was cultured for 2 days on the micropillar substrate, a fibrin solution (6.0 mg/mL fibrinogen and 2.0 U/mL thrombin) was pipetted on top of microvascular mesh and then the device was placed in fibrin solution. Another microvascular mesh on a micropillar substrate was flipped and placed in the fibrin solution, on top of the device. After 15 minutes of incubation at 37° C., microvascular meshes were embedded in fibrin gel and positioned onto the device surface (“Mesh” device, FIG. 18). The “Mesh” device was cultured in EGM-2 medium for approximate 4 hours prior to transplantation. As controls, a fibrin solution (6.0 mg/mL fibrinogen and 2.0 U/mL thrombin) without cells (“No cell” device) or containing a random mixture of ECs and NHDFs (ECs:NHDFs=9:1, cell number was same as in microvascular mesh, “Random” device) was gelled around chamber device and cultured for 2 days prior to transplantation.

The severe combined immune deficiency (SCID)-Beige mice (male and female, model number CBSCBG, 6-8 weeks old, Taconic Biosciences) were anesthetized using 2.5% (v/v) isofluorane in oxygen and maintained at the same rate throughout the procedure. Three ˜2.5 cm² squares were shaved (two on one flank and one on the opposite flank of the mouse) and sterilized with alternating scrubs of betadine and 70% (v/v) ethanol. A small ˜0.8 cm incision was created using scissors and a subcutaneous pocket was created through blunt dissection. This process was repeated on the opposite flank. The “Mesh” device was placed in subcutaneous pocket and sutured using 5-0 nylon. For controls, “No cell” and “Random” devices were also implanted.

Isolation of Rat Islets and Introduction of Rat Islets into Diffusion Chamber Devices

All animal procedures were approved by and performed according to the guidelines of the Institutional Animal Care and Use Committee at the Cornell University.

Sprague-Dawley rats (male, strain code 400, 250-275 g weight, Charles River) were used for harvesting islets. The bile duct was cannulated and the pancreas was distended by injection of 0.15% Liberase (Research Grade, Roche) in RPMI 1640 medium (Gibco). The pancreas was digested at 37° C. for 30 minutes. Digested pancreases were washed, filtered through a 450 μm sieve, and then suspended in a Histopaque 1077 (Sigma)/M199 medium (Gibco) gradient and centrifuged at 1700 g at 4° C. This gradient centrifugation step was repeated for obtaining purified islets. The islets were collected from the gradient and further isolated by a series of gravity sedimentations, in which each supernatant was discarded after 4 minutes of settling. Purified islets were cultured in RPMI 1640 medium overnight for further use. GFP-expressing islets were isolated from SD-Tg(UBC-EGFP)2BalRrrc rats (or SD-EGFP, male, 8-12 weeks old, Rat Resource and Research Center). Approximately 500˜1000 islets can be isolated from each rat.

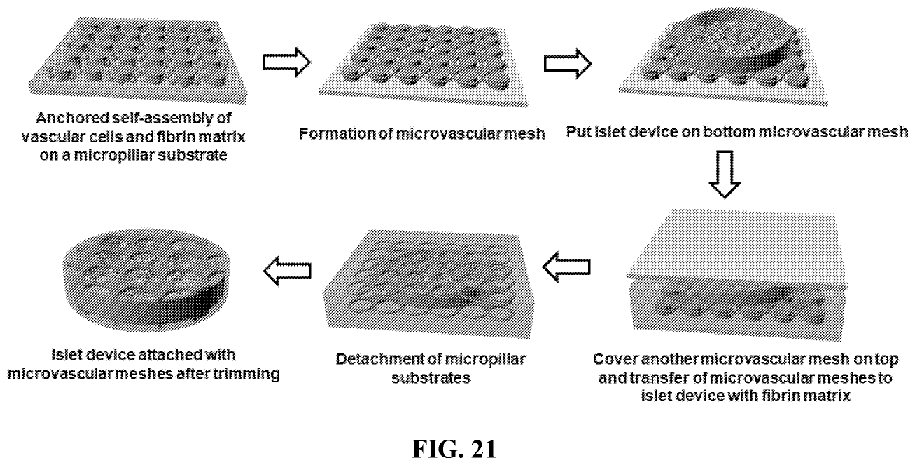

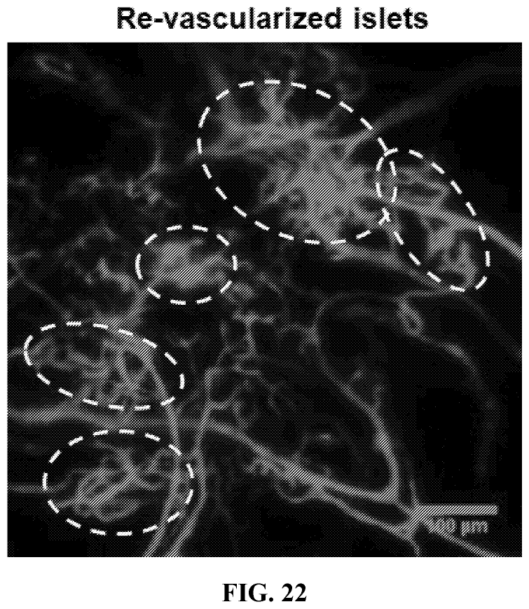

To load rat islets into the diffusion chamber device, a fibrin solution (6.0 mg/mL fibrinogen and 2.0 U/mL thrombin) containing rat islets (approximately 500 IEQ) was introduced into an 8 mm circular PDMS frame (inner diameter: 7 mm, thickness: 1 mm) with nylon grid at the bottom. A second nylon grid was placed on top of the PDMS frame and glued by gelling fibrin at 37° C. for 15 min. And then a fibrin solution (6.0 mg/mL fibrinogen and 2.0 U/mL thrombin) was pipetted on top of a HUVEC microvascular mesh on a micropillar substrate which had been cultured for 2 days in EGM-2 medium. The device containing rat islets was placed in the fibrin solution, and another HUVEC microvascular mesh on a micropillar substrate was flipped and placed in the fibrin solution, on top of the device. After 15 minutes of incubation at 37° C., the device, with HUVEC microvascular meshes on both sides, was fabricated (“Mesh” device, n=9, from 3 isolation and encapsulation experiments, FIG. 21). For “No cell” device (n=6, from 2 isolation and encapsulation experiments), a fibrin solution containing rat islets was loaded into a PDMS frame with a nylon grid at the bottom. Another nylon grid was placed on top of the PDMS frame and glued by gelling fibrin at 37° C. for 15 minutes. The “Random” device (n=6, from 2 isolation and encapsulation experiments) was prepared in the same way as “No cell” device except the random mixture of ECs and NHDFs in fibrin matrix had been cultured on the nylon grids for 2 days. Subcutaneous transplantation was performed as previously described for the empty diffusion chamber device without islets. In iPSC-EC and rat islet transplantation, n=5 in “No iPSC-EC” group, n=6 in “Random iPSC-EC” group, and n=6 in “Mesh iPSC-EC” group were from 2 isolation and encapsulation experiments.

### Evaluation of Hyperglycemia Correction in Diabetic Mice

To create insulin-dependent diabetic mice, healthy SCID-Beige mice were treated with freshly prepared streptozocin (STZ) solution (120 mg/kg mouse) twice via intraperitoneal injection. The blood glucose level of all the mice was retested prior to transplantation. Only mice whose non-fasting blood glucose level was above 350 mg/dL were considered diabetic. Mice were randomized among control and treatment groups to keep blood glucose levels at a similar level for all groups at the beginning of transplantation.

Non-fasting blood glucose levels were monitored twice during the first week and then once a week following the transplant surgery. Blood glucose measurements were randomly performed by blinded and non-blinded personnel. A small droplet of blood was collected from tail vein and glucose concentration was measured with a commercial glucometer (Clarity Plus, Diagnostic Test Group). Mice with non-fasting blood glucose levels below 200 mg/dL were considered normoglycemic. After retrieving the devices from diabetic mice non-fasting blood glucose was monitored for one more week. The retrieved devices were fixed in 10% formalin, embedded in paraffin, and sectioned. Hematoxylin/eosin staining and immunostaining of the sections were performed.

Intraperitoneal glucose tolerance test (IPGTT) was measured one month and three months after surgery. The mice were fasted for approximately 16 hours. Glucose was administered via intraperitoneal injection (2.0 g/kg mouse), and blood glucose levels were measured at the indicated times.

### Mouse Blood Vessel Perfusion

To image anastomoses between mouse and human blood vessels, two types of lectins (100 μL each) were injected into the mouse through the tail vein after 14 days of transplantation. Green fluorescein Ulex Europaeus Agglutinin I (UEA-I) lectin (2 mg/mL) specifically bound to human endothelial cells (Baranski et al., “Geometric Control of Vascular Networks to Enhance Engineered Tissue Integration and Function,” Proc. Natl. Acad. Sci. U.S.A. 110:7586-7591 (2013), which is hereby incorporated by reference in its entirety). Red DyLight 594 Labeled Griffonia Simplicifolia lectin I (GSL-I) isolectin B₄ (1 mg/mL) specifically bound to mouse endothelial cells (Kang et al., “Bioengineered Human Vascular Networks Transplanted Into Secondary Mice Reconnect With the Host Vasculature and Re-Establish Perfusion,” Blood 118:6718-6721 (2011), which is hereby incorporated by reference in its entirety). Mice were euthanized, and devices were harvested and fixed in 10% (v/v) formalin. Blood vessels were imaged with a laser scanning confocal microscope (LSCM, FLUOVIEW FV1000, Olympus). To evaluate the earliest time point that blood-perfused human vasculatures were formed and anastomosed with mouse vascular system, microvascular meshes of HUVEC-GFP/NHDF (9:1, “Mesh” device) and random mixture of HUVEC-GFP/NHDF (9:1, “Random” device) were subcutaneously implanted, and mice were perfused with lipophilic carbocyanine dye DiI as previously describe (Li et al., “Direct Labeling and Visualization of Blood Vessels With Lipophilic Carbocyanine Dye DiI,” Nat Protoc 3:1703-1708 (2008), which is here by incorporated by reference in its entirety) at different time points (Day 4, 7 and 10). Briefly, a butterfly needle was inserted into left ventricle of the mouse, and 2 mL of PBS, 10 mL of DiI solution, and 10 mL of 4% (v/v) paraformaldehyde solution were perfused sequentially at the speed of 2 mL/minute using a syringe pump. Immediately after perfusion, devices were retrieved and imaged using laser scanning confocal microscope. The overlap of green HUVEC-GFP and red dye DiI indicated that human vasculatures were blood-perfused and connected with mouse vascular system. To visualize the vascularization of implanted rat islets, mouse blood vessels were perfused with dye DiI and retrieved devices were imaged in the same way.

### Immunohistochemistry and Image Analysis

Microvascular meshes cultured for 2 days in vitro were fixed in 10% formalin, permeabilized with 0.2% (v/v) Triton X-100, and blocked with 2% (w/v) bovine serum albumin (BSA) solution. Human CD31 was detected with a primary mouse anti-human CD31 antibody (1:200, eBioscience) followed by a secondary Alexa Fluor™ 488 goat anti-mouse antibody (1:400, Invitrogen) or Alexa Fluor™ 555 donkey anti-mouse antibody (1:400, Invitrogen). F-actin was stained with Texas-Red™-X or Alexa Fluor™ 488 phalloidin (Invitrogen). Cell nuclei were counter-stained with Hoechst (Invitrogen). The images were obtained using a fluorescence microscope (EVOS, AMG).

Laser scanning confocal microscope (LSCM, FLUOVIEW FV1000, Olympus) was used to image 3D structure of microvascular mesh. Images were acquired using FV10-ASW2.0 software (Olympus). The cross-sectional images were analyzed with Fiji ImageJ.

Devices retrieved from mice were fixed in 10% formalin, processed, embedded, and sectioned for immunohistochemistry. The primary antibodies were rabbit anti-human CD31(1:200, Sigma), goat anti-mouse CD31 (1:200, R&D systems), and a-smooth muscle actin antibody conjugated with Cy3 (1:200, Sigma). The secondary antibodies were Alexa Fluor™ 594 donkey anti-rabbit antibody (1:400, Invitrogen), Alexa Fluor™ 488 donkey anti-rabbit antibody (1:400, Invitrogen), and Alexa Fluor™ 488 donkey anti-goat antibody (1:400, Invitrogen). After washing with PBS, the slides were mounted in Fluoroshield with DAPI (Sigma) and imaged with a fluorescence microscope (EVOS, AMG).

On hematoxylin/eosin staining images, human or mouse blood vessels were identified by luminal structures with erythrocytes inside. The vessel number was quantified by counting individual vessels within the interfacial area between the device and panniculus carnosus muscle. The vessels density (vessels/mm²) was calculated by dividing the total vessel number by the interfacial area. The area percentage of vessels was calculated by dividing the total area of erythrocyte-containing luminal structures by the interfacial area. To quantify the percentage of blood-perfused human vessels, histological slides of retrieved devices (10 days after transplantation) were stained with human CD31 and α-SMA antibodies. The blood-perfused human vasculatures were identified by vessel cross-sections which had luminal shape (with erythrocytes inside) and were positively stained by both human CD31 and α-SMA antibodies.

For immunohistochemical detection of rat insulin, histological sections were stained with rabbit anti-rat insulin (1:200, abcam). The secondary antibody was Alexa Fluor™ 594 donkey anti-rabbit antibody (1:400, Invitrogen). Cell nuclei were stained with Hoechst (Invitrogen).

### Example 1—Fabrication and Function of the Microvascular Meshes

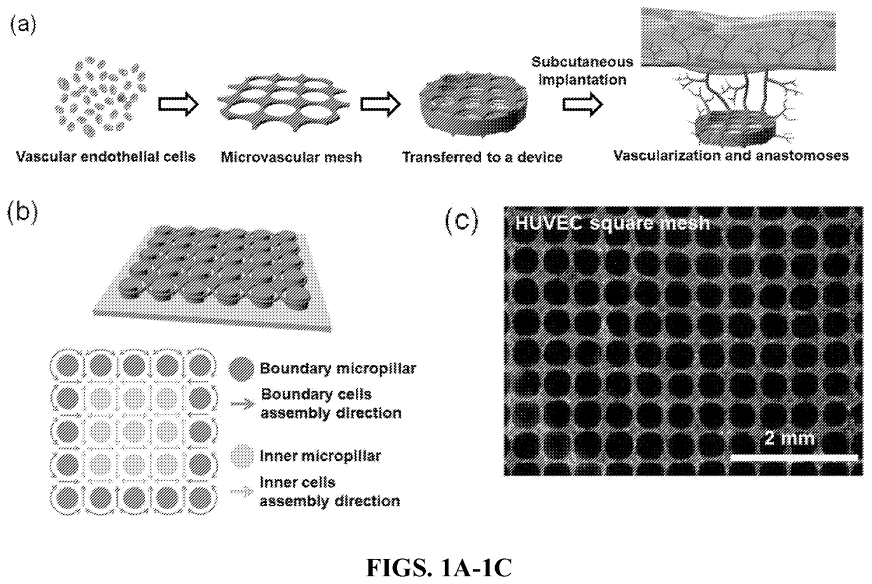

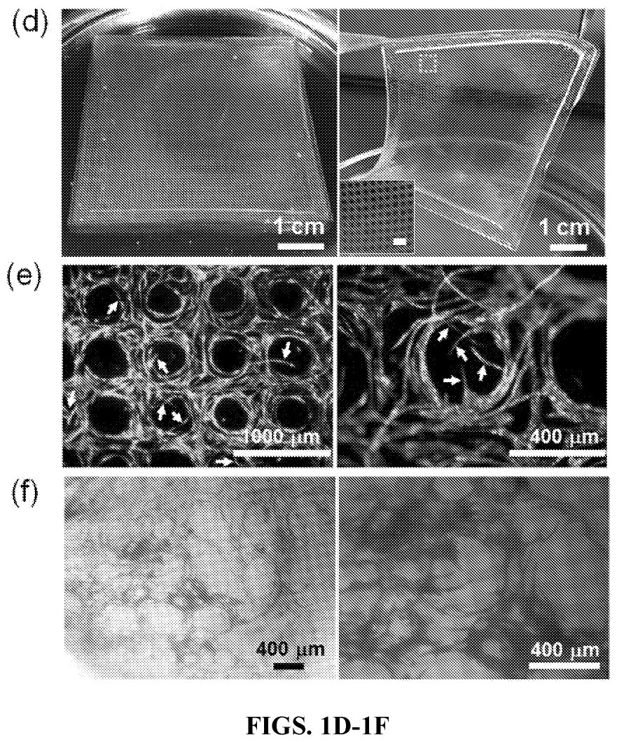

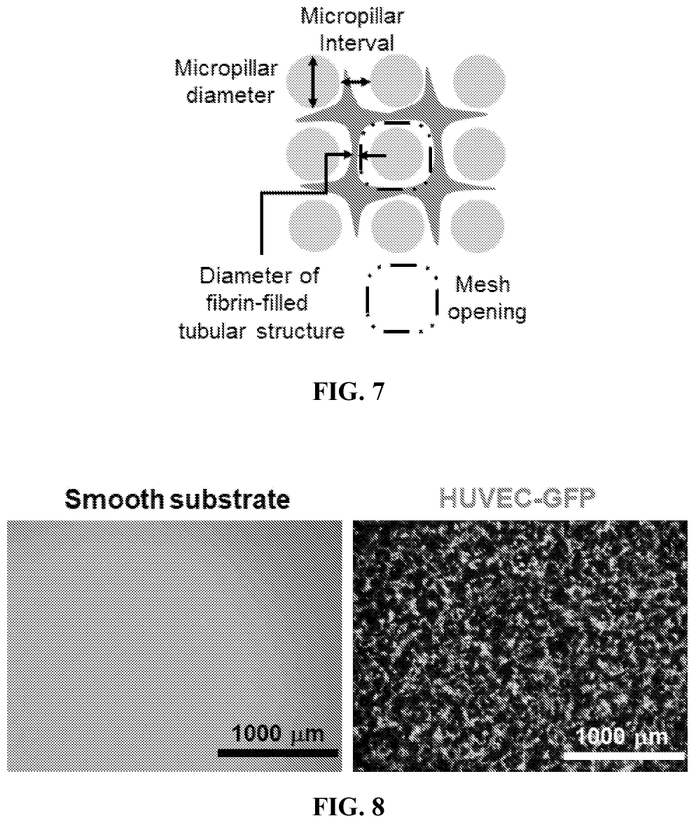

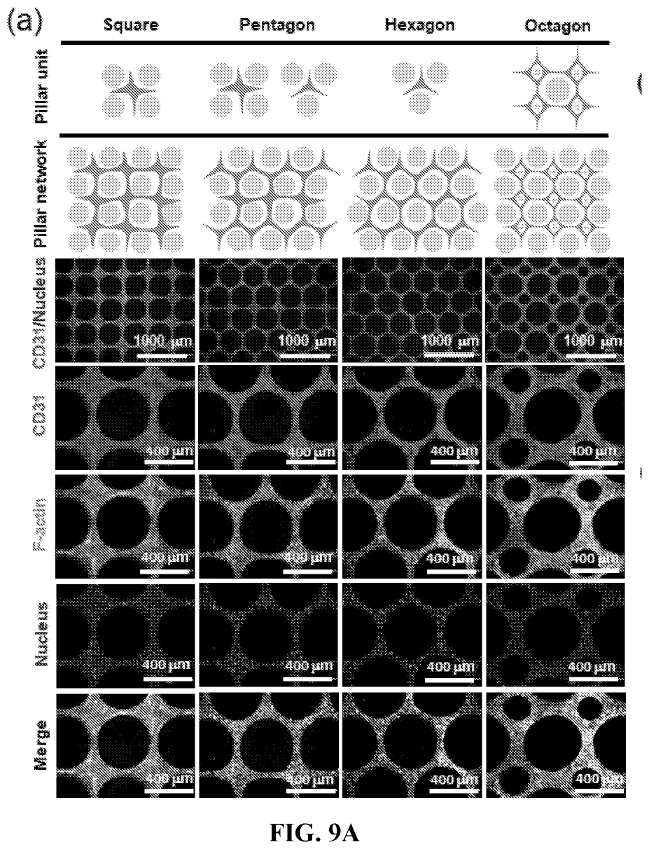

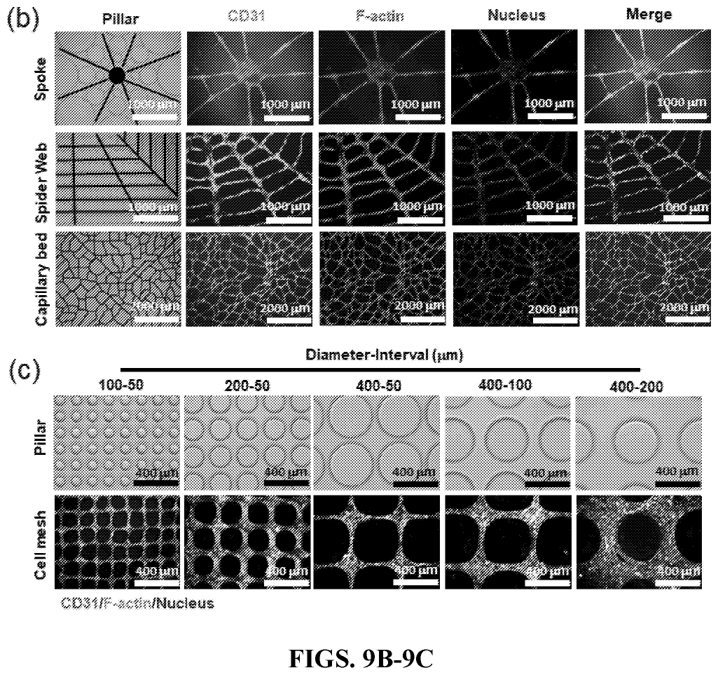

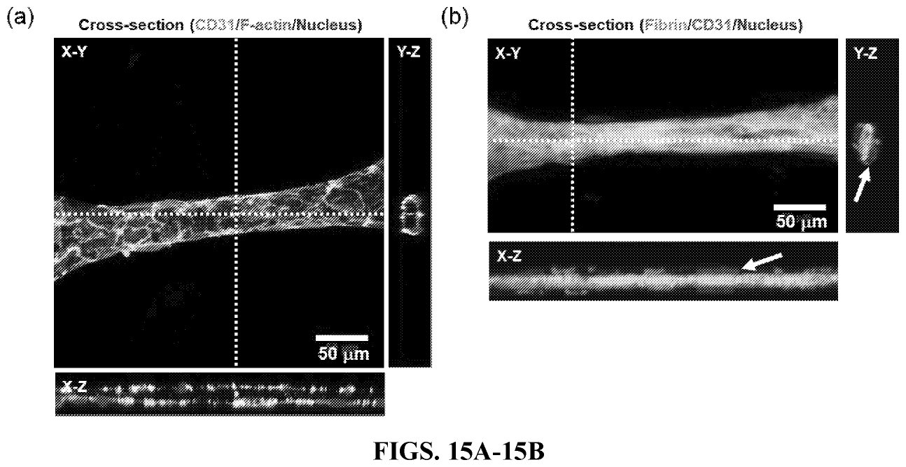

To achieve rapid and functional vascularization around a cellular device, one straightforward approach is to attach a pre-formed vascular structure with sufficient density and resolution to the device; upon implantation (e.g., in subcutaneous space) the vascular structure induces angiogenesis and promotes anastomoses with host vasculature (FIG. 1A). To fabricate such a transferrable vascular structure in a controlled manner, an Anchored Self-Assembly (ASA) strategy was developed. The key to the ASA is a cell organization process on the micropillar substrate where the inner micropillars serve as a geometric template to guide the cell organization into long-range ordering and the boundary micropillars play an “anti-contraction” effect to prevent the shrinkage of the mesh structure, leading to a stable and ordered microvascular mesh (FIG. 1B). It was found that HUVECs, together with a fibrin matrix, self-assembled into an almost defect-free square mesh after 2 days of culture on the micropillar substrate (FIG. 1C). In contrast, HUVEC structures remained random when cultured on a smooth substrate without micropillars (FIG. 8). The mesh geometry and dimension could be precisely controlled by adjusting the size and arrangement of micropillars. Simple geometries such as square, pentagon, hexagon, and octagon (FIG. 9A), as well as complex structures that resembled spoke, spider web, and natural capillary bed (FIG. 9B) are illustrated herein. Using the square network as an example, the diameter of the fibrin-filled tubular structure (FIG. 15B) could be controlled from approximately 15 μm to 133 μm, the size of mesh opening from 150 μm to 467 μm, and density from 2 openings/mm² to 44 openings/mm² (FIG. 9C, Table 1, FIG. 7).

TABLE 1
Characterization of the Diameter of Fibrin-Filled Tubular Structure, and the Size
and Density of Mesh Opening on Different Micropillar Substrates.

| Micropiller Diameter-Inteival | Diameter of Fibrin- | Mesh Opening Size | Mesh Opening Density |
|---|---|---|---|
| (μm) | Filled TubuLar Structure (μm) | (μm) | (openings/mm²) |
| 100-50 | 15.0 ± 0.5 (n = 75) | 150.0 ± 0.5 (n = 75) | 44 |
| 200-50 | 36.9 ± 0.5 (n = 185) | 213.1 ± 0.5 (n = 185) | 16 |
| 400-50 | 23.1 ± 0.6 (n = 49) | 427.0 ± 0.6 (n = 49) | 5 |
| 400-100 | 51.6 ± 1.8 (n = 48) | 448.4 ± 1.8 (n = 48) | 4 |
| 400-200 | 133.0 ± 1.5 (n = 131) | 467.0 ± 1.5 (n = 131) | 2 |

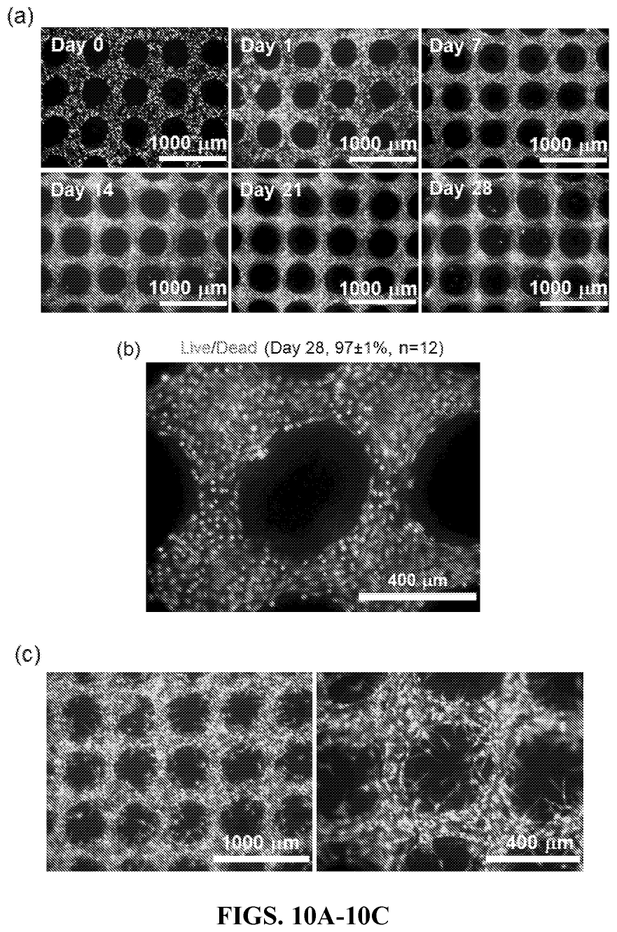

The ASA approach is scalable and the microvascular meshes can be lifted from the micropillar substrate for transferring. A 5×5 cm, free-standing, square mesh was fabricated from HUVECs (FIG. 1D). The meshes were stable and cells were viable on micropillar substrate for at least 4 weeks (FIG. 10A and FIG. 10B) but rapidly generated sprouts after being transferred and embedded in a fibrin matrix (FIG. 1E and FIG. 10C). The meshes promoted dense, functional vascularization on a diffusion chamber after 2 weeks of subcutaneous implantation in SCID-Beige mice (FIG. 1F). Blood-perfused vessels maintained square-like network with numerous newly-formed sprouts, similar to angiogenic sprouting in vitro. It should be noted that microvascular meshes were constantly re-molded during the development of vascularization and anastomoses, and therefore, the original shapes of mesh network were not always preserved.

### Example 2—Simulation and Characterization of the Microvascular Meshes

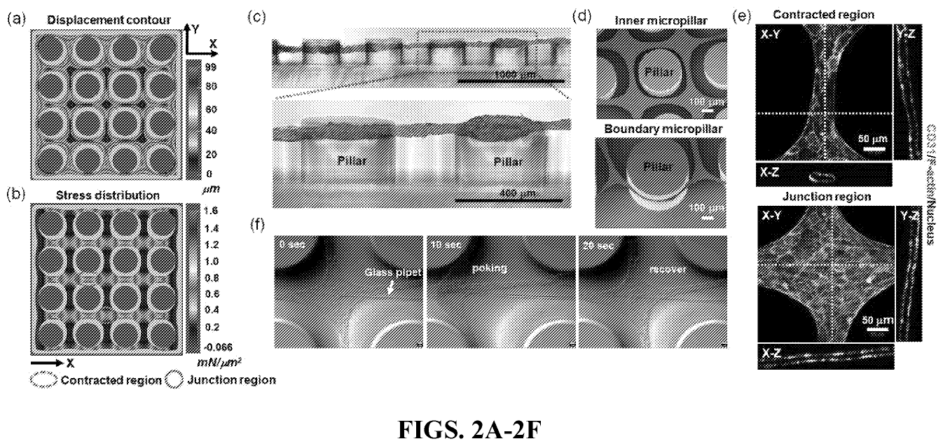

To better understand the ASA process, a finite element simulation of the cellular assembling process and further characterization of the microvascular mesh was performed. The simulation, which considers the contractile action of the cells on the fibrin matrix, generated an in-plane displacement contour plot of organized mesh structure (FIG. 2A), and stress and strain distribution (FIG. 2B and FIG. 11) on the micropillar substrate. The assembling cells and matrix gradually stopped being in contact with the inner micropillars, and finally only the boundary micropillars were in contact with cells and supported the entire mesh structure (FIG. 2A). The cells and matrix close to boundary micropillars sustained contraction from one direction, while those away from the boundary experienced contraction from all directions, and exhibited higher stresses in the contracted region than in the junction region along both the X (Cauchy stress component 11 in FIG. 2B) and Y (Cauchy stress component 22 in FIG. 11A) directions.

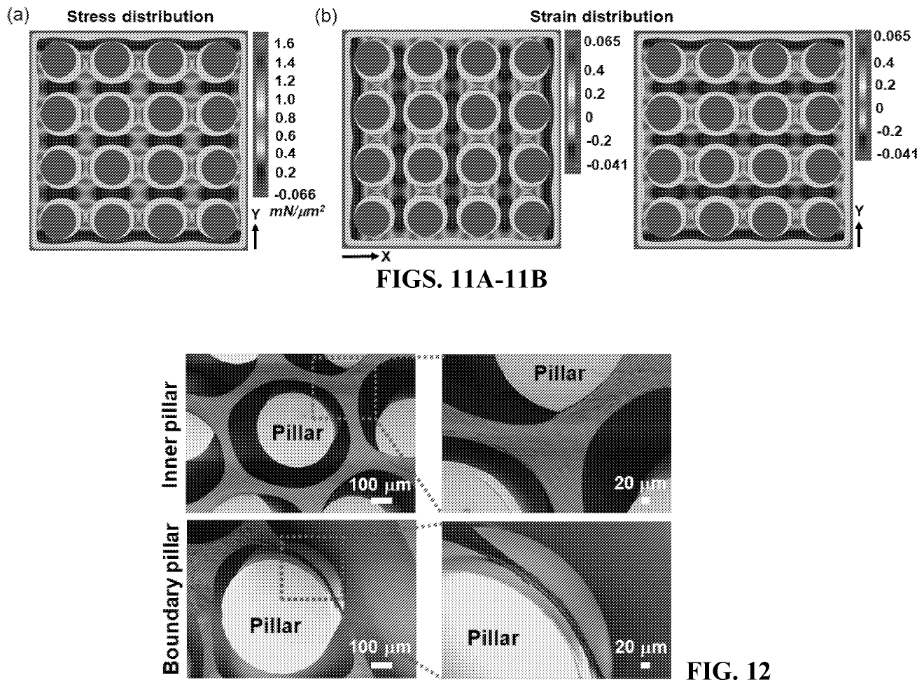

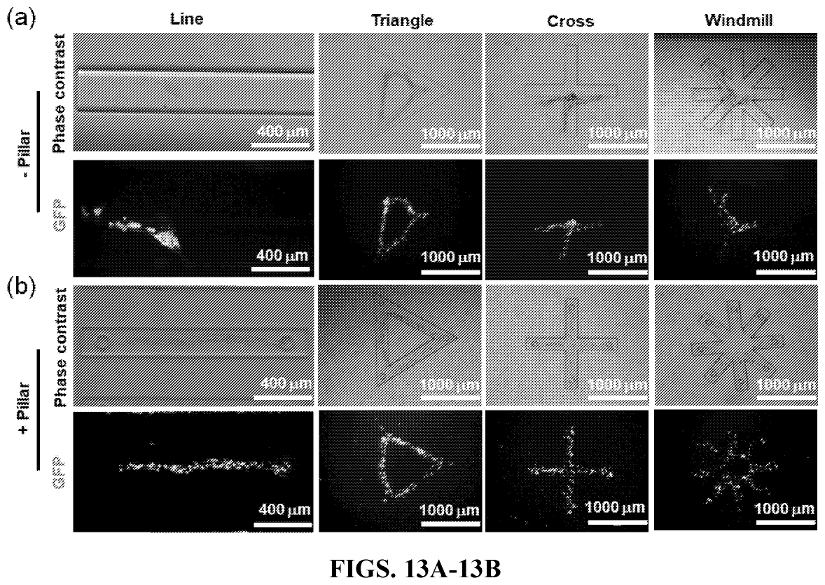

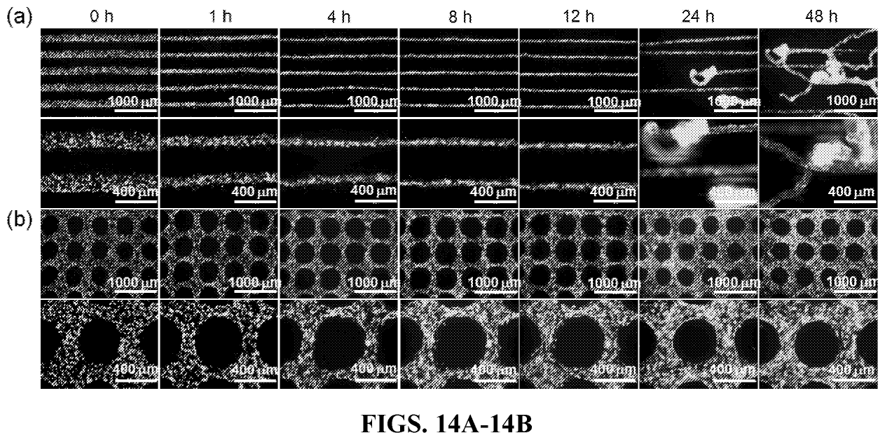

Interestingly, cross-sectional images showed indeed the microvascular meshes were tightened and suspended between micropillars rather than settling on the bottom (FIG. 2C), consistent with the simulation results. Scanning electron microscope (SEM) images (FIG. 2D and FIG. 12) also confirmed that the HUVEC mesh hung among inner micropillars with more contracted regions between junctions and the whole mesh was prevented from shrinking by boundary micropillars. Control experiments further supported that the formation of a stable cell construct is not through a simple space-filling mechanism alone but highly reliant on the micropillars. For example, when HUVEC/fibrin mixture was introduced into grooves with different shapes (e.g., linear, triangle, cross, and windmill) without micropillars inside, cell/fibrin mixture formed structures that were only temporarily stable and all shrank into clumps within 48 hours (FIG. 13A and FIG. 14A) due to intrinsic cellular contraction. In contrast, when micropillars were present inside, cells self-organized into different structures that corresponded to the shapes of the grooves (FIG. 13B and FIG. 14B).

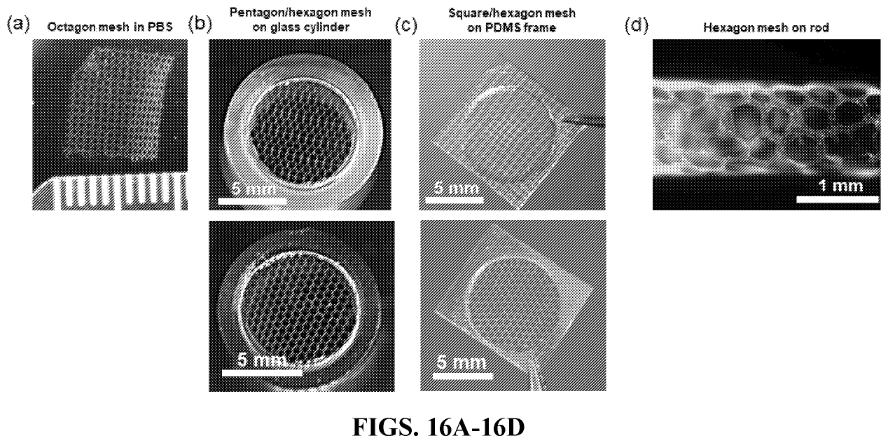

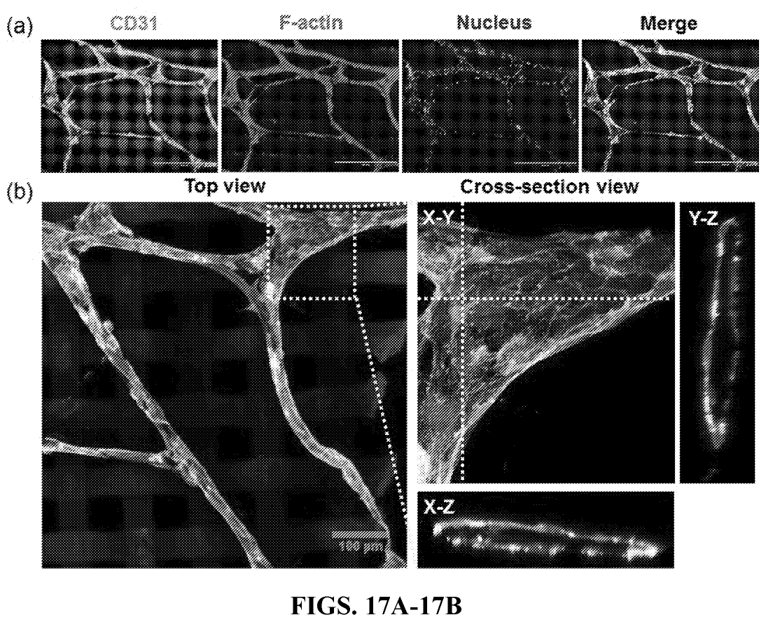

Confocal images showed that the HUVEC mesh had continuous and interconnected tubular structures (FIG. 2E and FIG. 15A) in both contracted and junction regions. Further staining showed that the interior of the tubular structure was filled with fibrin on which HUVECs coalesced and adhered (FIG. 15B). This self-assembled, cell/fibrin composite structure was consistent with earlier reports (Baranski et al., “Geometric Control of Vascular Networks to Enhance Engineered Tissue Integration and Function,” Proc. Natl. Acad. Sci. U.S.A. 110:7586-7591 (2013) and Chaturvedi et al., “Patterning Vascular Networks In Vivo for Tissue Engineering Applications,” Tissue Eng. Part C 21:509-517 (2015), each of which is hereby incorporated by reference in its entirety) and resembled the de novo formation of primitive vasculatures that also involves coalescence of endothelial progenitor cells and subsequent lumen formation (Bersini et al., “Cell-Microenvironment Interactions and Architectures in Microvascular Systems,” Biotechnol. Adv. 34:1113-1130 (2016) and Raghavan et al., “Geometrically Controlled Endothelial Tubulogenesis in Micropatterned Gels,” Tissue Eng. Part A 16:2255-2263 (2010), each of which is incorporated by reference in its entirety). Another important characteristic of the ASA-enabled microvascular meshes is their mechanical robustness. The meshes were elastic and resilient; they even withstood poking with a 6-□m glass pipette. As shown in FIG. 2F, the mesh was displaced approximately 150 μm without any visible damage and then recovered to its original position when the pipette was withdrawn. This remarkable mechanical property allowed manipulation and transfer of the mesh to different substrates (FIG. 16) without affecting the integrity and fibrin-filled tubular structures of the mesh (FIG. 17).

### Example 3—Enhanced Vascularization of Subcutaneously Implanted Devices in SCID-Beige Mice

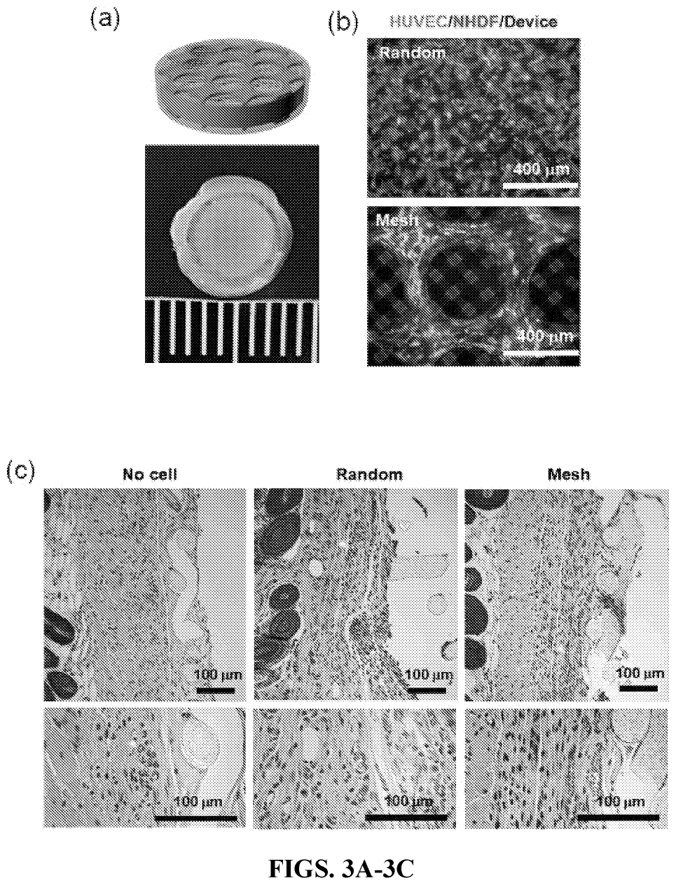

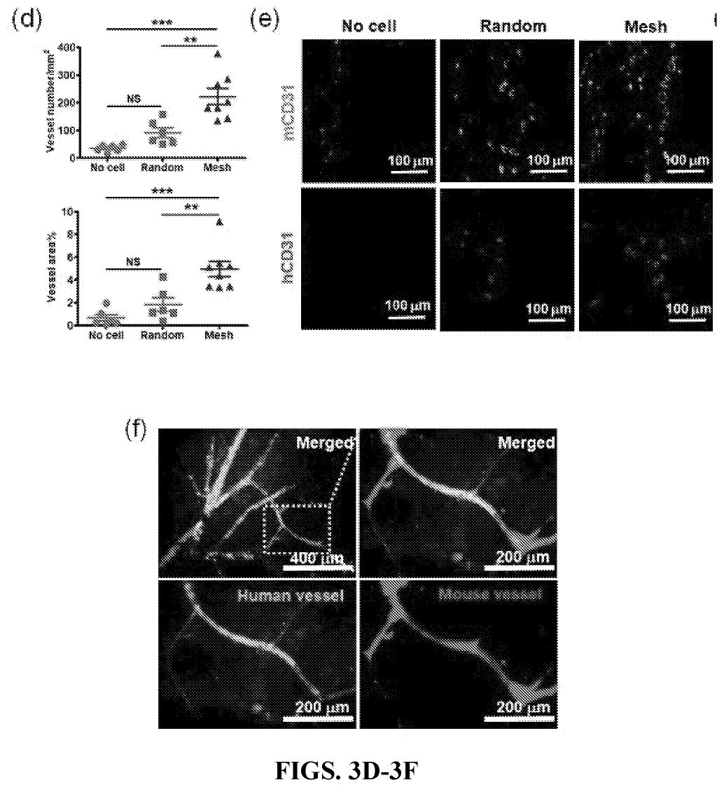

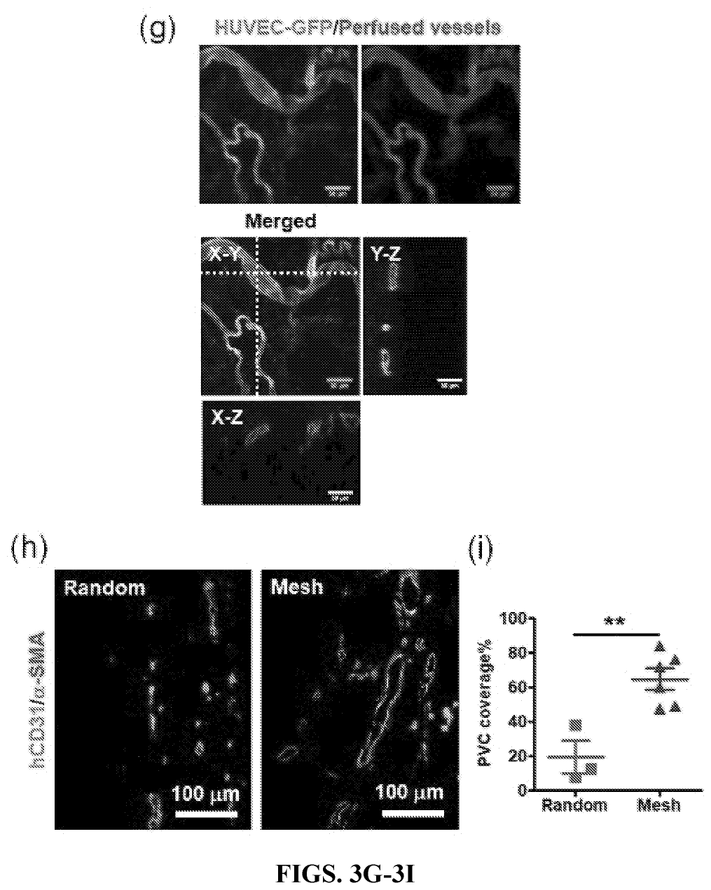

To quantitatively investigate how microvascular meshes enhanced vascularization, HUVEC meshes were compared with a random HUVEC/fibrin mixture. In both cases, normal human dermal fibroblasts (NHDFs) were added (HUVECs:NHDFs=9:1) to support and enhance vessel formation (Nakatsu et al., “Angiogenic Sprouting and Capillary Lumen Formation Modeled by Human Umbilical Vein Endothelial Cells (HUVEC) in Fibrin Gels: The Role of Fibroblasts and Angiopoietin-1,” Microvasc. Res. 66:102-112 (2003), which is hereby incorporated by reference in its entirety). Microvascular meshes or random cell mixture were attached to diffusion chamber devices using a fibrin gel (“Mesh” device (n=8) and “Random” device (n=6); FIGS. 3A, 3B; FIG. 18). Devices without any cells (“No cell” device (n=6)) were used as an additional control. All devices were then implanted into the subcutaneous space of SCID-Beige mice. Subcutaneous space is a poorly-vascularized site but has many advantages for cell replacement therapies including relatively easy accessibility, minimal invasiveness, and potentially high transplant capacity (Sakata et al., “Strategy for Clinical Setting in Intramuscular and Subcutaneous Islet Transplantation,” Diabetes Metab. Res. Rev. 30:1-10 (2014), which is hereby incorporated by reference in its entirety).

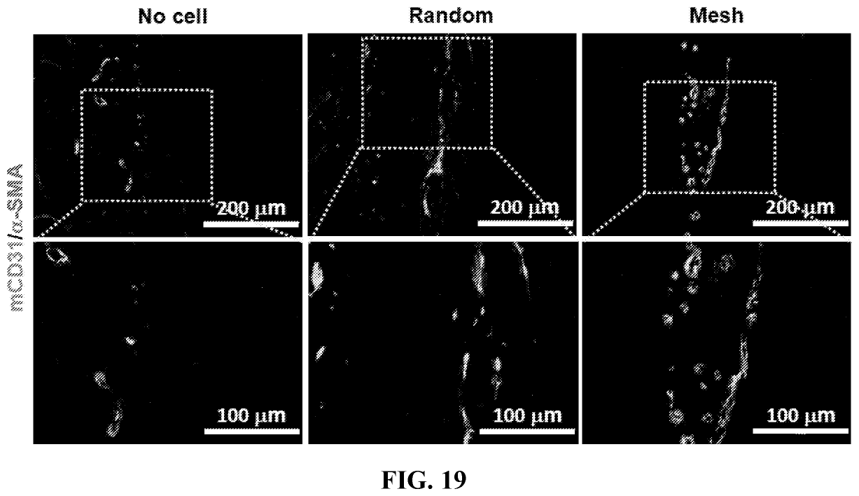

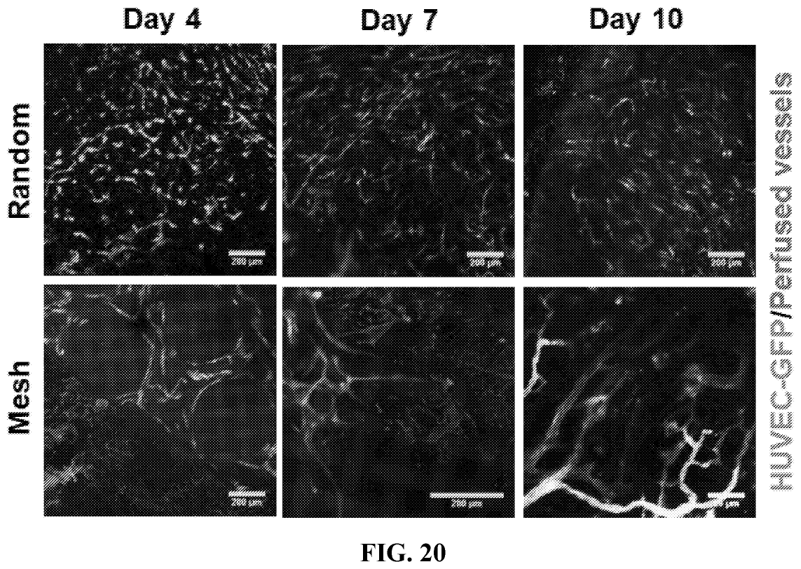

The devices were retrieved, and vascularization was compared after 2 weeks of implantation. Histological hematoxylin/eosin (H&E) staining (FIG. 3C) and quantification of blood vessels (FIG. 3D) surrounding the devices revealed a significantly higher vascularization in the “Mesh” device, compared to the “No cell” and “Random” devices. These vasculatures were mature as indicated by α-smooth muscle actin (α-SMA) staining (FIG. 19). Interestingly, positive immunostainings for both human CD31 (FIG. 3E; red) and mouse CD31 (FIG. 3E; green) seemed to suggest that newly formed vessels (in both “Random” and “Mesh” devices) were chimeric in nature, indicating the occurrence of anastomoses and vascular re-modeling during vascularization. To further demonstrate that generated vessels were functionally connected with mouse vasculature and to confirm the presence of anastomoses, mice were perfused with the “Mesh” devices through tail vein injections using two lectins. The first lectin was a green fluorescent Ulex Europaeus Agglutinin I (UEA-I) lectin that specifically bound to human ECs (Baranski et al., “Geometric Control of Vascular Networks to Enhance Engineered Tissue Integration and Function,” Proc. Natl. Acad. Sci. U.S.A. 110:7586-7591 (2013), which is hereby incorporated by reference in its entirety) and the second one was a red Griffonia Simplicifolia lectin I (GSL-1) isolectin B₄ that was specific for mouse ECs (Kang et al., “Bioengineered Human Vascular Networks Transplanted Into Secondary Mice Reconnect With the Host Vasculature and Re-Establish Perfusion,” Blood 118:6718-6721 (2011), which is hereby incorporated by reference in its entirety). The overlap of human- and mouse-specific lectin binding (FIG. 3F) supported that HUVEC mesh promoted not only neovascularization but also anastomoses. Furthermore, to evaluate the earliest time point that blood-perfused human vasculatures were formed and anastomosed with mouse vascular system, microvascular meshes of HUVEC-GFP/NHDF (9:1, “Mesh” device) and a random mixture of HUVEC-GFP/NHDF (9:1, “Random” device) were subcutaneously implanted, and lipophilic carbocyanine dye DiI (Li et al., “Direct Labeling and Visualization of Blood Vessels With Lipophilic Carbocyanine Dye DiI,” Nat Protoc 3:1703-1708 (2008), which is hereby incorporated by reference in its entirety) was perfused into mouse vascular system at different time points (Day 4, 7 and 10, FIG. 20 and FIG. 3G). At Day 10, in “Mesh” device, we found that human vasculatures were functional and connected with mouse vascular system since green color of HUVEC-GFP overlapped with red color of perfused dye DiI. However, in “Random” device, the formation of blood-perfused human vessels was not observed under the confocal microscope at Day 10 (FIG. 20). To quantify the percentage of blood-perfused human vessels, histological slides of retrieved “Random” and “Mesh” devices were stained with human CD31 and a-SMA antibodies. The blood-perfused human vasculatures were identified by vessel cross-sections which had luminal shape (with erythrocytes inside) and were positively stained by both human CD31 and a-SMA antibodies (FIG. 3H). The percentage of perfused human vessels is 19±9% (n=3) in “Random” device and 65±6% (n=6) in “Mesh” device (FIG. 31). All the results, taken together, substantiate that ASA-enabled, transferrable microvascular meshes have potential use for vascularization of cell delivery devices.

### Example 4—Correction of Chemically Induced Diabetes in SCID-Beige Mice Using Rat Islets

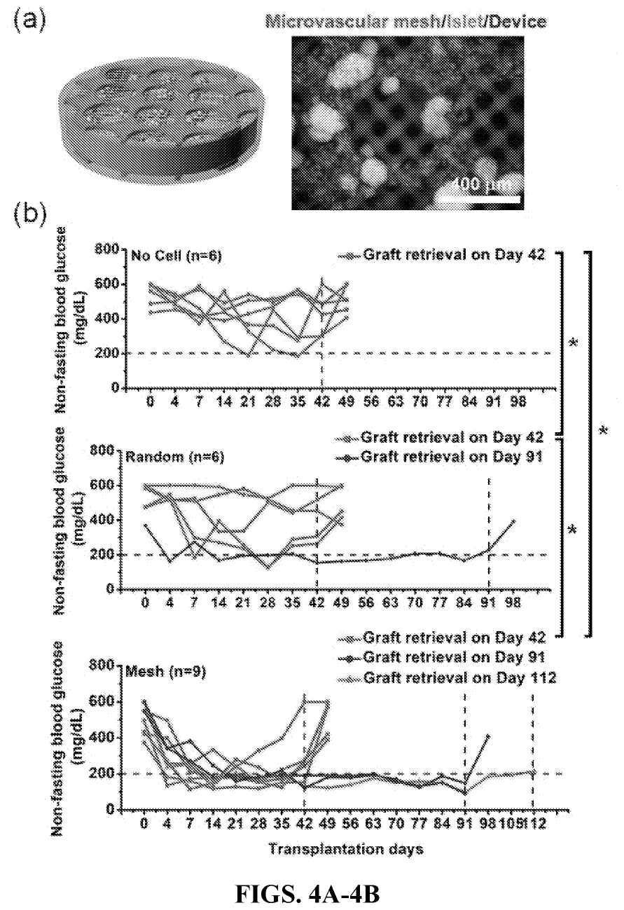

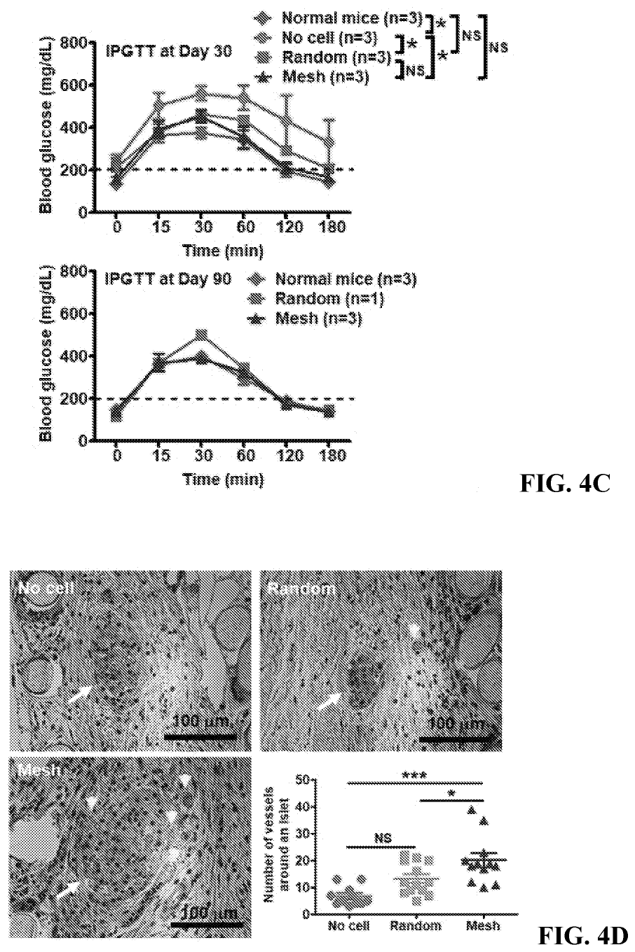

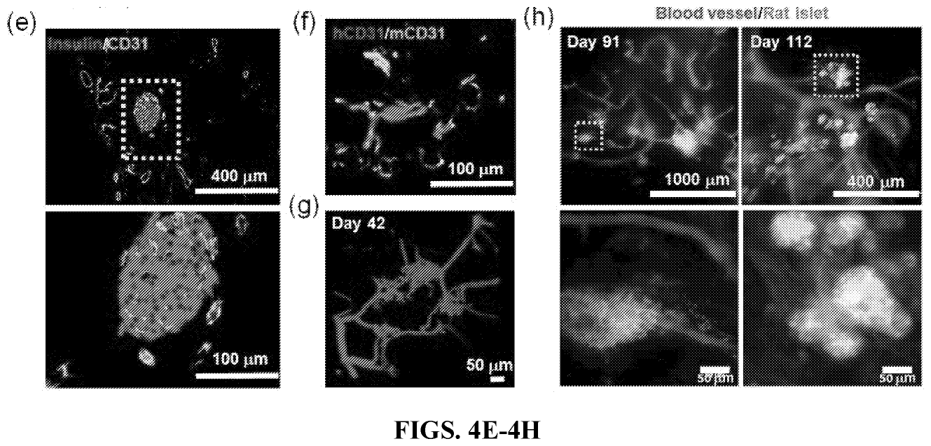

It was next investigated whether microvascular meshes could be used to improve cell replacement therapy for T1D. Rat islets were loaded in diffusion chamber devices (FIG. 4A; FIG. 21), attached microvascular meshes (HUVECs:NHDFs=9:1) to the devices using a fibrin gel and transplanted the constructs subcutaneously in SCID-Beige mice with STZ-induced diabetes. The device had an open structure with pore size ˜70 μm, smaller than the average islet size (˜150 μm). Each device contained ˜500 islet equivalents (IEQ) (Ricordi et al., “Islet Isolation Assessment in Man and Large Animals,” Acta Diabetol. Lat. 27:185-195 (1990), which is hereby incorporated by reference in its entirety). To better visualize islets GFP rat islets were loaded in two “Mesh” devices (FIG. 4A). Devices encapsulating similar number of islets but with random HUVECs/NHDFs (“Random” device) or without any HUVECs/NHDFs (“No cell” device) were used as controls. After transplantation, non-fasting blood glucose (BG) concentration decreased in some mice in all three groups. However, mice in the “Mesh” group (n=9) had significantly better BG control compared to the “No cell” (n=6) and “Random” groups (n=6) during 42 days of transplantation. In addition, more mice in the “Mesh” group became normoglycemic (<200 mg/dL) and also maintained normoglycemia for a longer time (FIG. 4B and Table 2). Although the earliest formation of blood-perfusable, anastomosed vasculature was observed on Day 10 in empty “Mesh” device (no islet, FIG. 3G and FIG. 20), 2 out of 9 mice in the “Mesh” group had normal BG as early as 4 days post transplantation (Table 2). This may suggest that the viability and function of islets could benefit from the paracrine secretion/signaling of HUVECs microvascular meshes and recruitment of endogenous vessels during early days of transplantation. The number of cured mice in the “Mesh” group increased to 7 mice at 14 days post transplantation (Table 2) after functional human vasculatures and anastomoses with mouse vascular system had been established. Grafts were retrieved for characterization at Day 42 from most mice except 1 normoglycemic mouse from the “Random” group and 3 normoglycemic mice from the “Mesh” group which were kept longer until Day 91 or Day 112. After retrieval, the BG increased in all mice, demonstrating the anti-diabetic effect of the devices. An intraperitoneal glucose tolerance test (IPGTT) was performed at 1 month and 3 months post-transplantation (FIG. 4C). The “Mesh” group showed a better glucose response than “No cell” group, and the glucose responsiveness was similar to healthy non-diabetic mice at both time points.

TABLE 2
The Summary of the Number of Cured Mice (Non-Fasting Blood Glucose <200
mg/dL) in No Cell, Random, and Mesh Devices During 112 Days of Transplantation.

| Transplantation | No cell | Random | Mesh |
|---|---|---|---|
| days | (cured mice/total mice) | (cured mice/total mice) | (cured mice/total mice) |
| 4 | 0/6 | 1/6 | 2/9 |
| 7 | 0/6 | 1/6 | 3/9 |
| 14 | 0/6 | 1/6 | 7/9 |
| 21 | 1/6 | 1/6 | 5/9 |
| 28 | 0/6 | 3/6 | 7/9 |
| 35 | 1/6 | 0/6 | 6/9 |
| 42 | 0/6 (Devices retrieved) | 1/6 (Devices retrieved | 4/9 (Devices retrieved |
| | | from 5 mice) | from 6 mice) |
| 49 | | 1/1 | 3/3 |
| 56 | | 1/1 | 3/3 |
| 63 | | 1/1 | 3/3 |
| 70 | | 0/1 | 3/3 |
| 77 | | 0/1 | 3/3 |
| 84 | | 1/1 | 3/3 |
| 91 | | 0/1 (Device retrieved) | 3/3 (Devices retrieved |
| | | | from 2 mice) |
| 98 | | | 1/1 |
| 105 | | | 1/1 |
| 112 | | | 0/1 (Device retrieved |

The devices retrieved at Day 42 were evaluated histologically for vascularization (FIG. 4D). Although viable islets with normal morphology were found in all three groups, islets in implants from the “Mesh” group were surrounded by significantly more blood vessels than the two control groups, consistent with the diabetes correction results (FIG. 4B). Although NHDFs were incorporated with HUVECs to promote the maturation of functional vessels, a few erythrocytes were observed to leak out of immature vessels (hemorrhage) in histological staining. To further enhance the maturation of newly formed vessels, mesenchymal stem cells could be used as supporting cells (Koike, N. et al., “Tissue Engineering: Creation of Long-Lasting Blood Vessels,” Nature 428:138-139 (2004), which is hereby incorporated by reference in its entirety). Additional immunostaining (FIG. 4E) confirmed that islets in the “Mesh” group were functional with positive insulin staining and were also highly vascularized (CD31 staining) both externally and internally. Similar to the results with empty devices (no islet, FIGS. 3E, 3F), positive overlapping staining with both human and mouse CD31 antibodies demonstrated anastomoses between human and mouse vessels (FIG. 4F). Moreover, to better visualize the connection between vasculatures inside rat islets and surrounding mouse vessels in mice from the “Mesh” group, whole mouse perfusions were performed prior to retrievals, at Day 42, Day 91, and Day 112, with a fluorescent lipophilic carbocyanine dye DiI (Li et al., “Direct Labeling and Visualization of Blood Vessels With Lipophilic Carbocyanine Dye Dil,” Nat Protoc 3:1703-1708 (2008), which is hereby incorporated by reference in its entirety). Confocal and fluorescent images of the perfused devices clearly showed interconnected, 3D structure of vasculatures in transplanted islets (FIG. 4G and FIG. 22 for the device from Day 42 retrieval, and FIG. 4H for the device with GFP rat islets from Day 91 and Day 112 retrievals). Together, these results confirmed the effectiveness of transferrable microvascular meshes in promoting re-vascularization of donor islets and maintaining normoglycemia for up to 3 months in diabetic mice.

### Example 5—Microvascular Meshes from Human iPSC-ECs

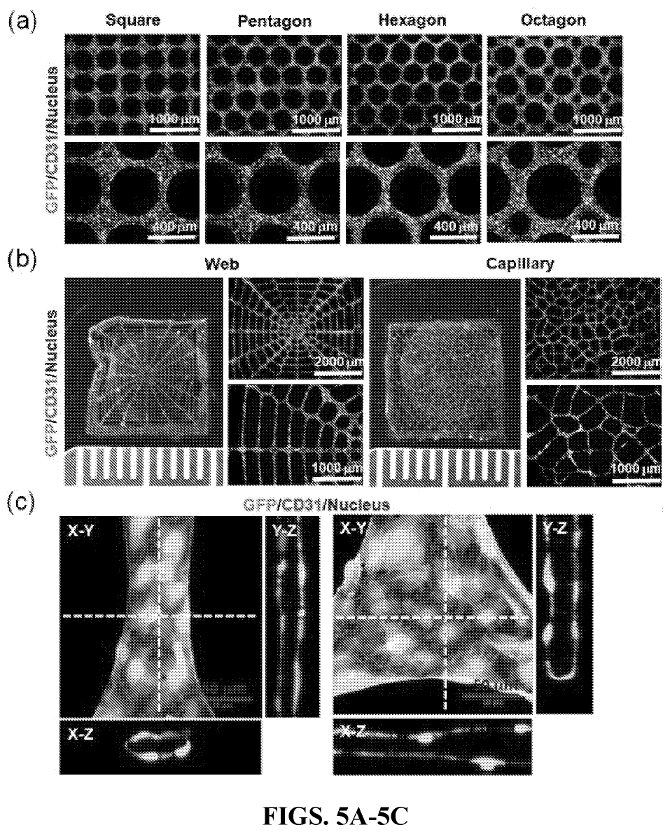

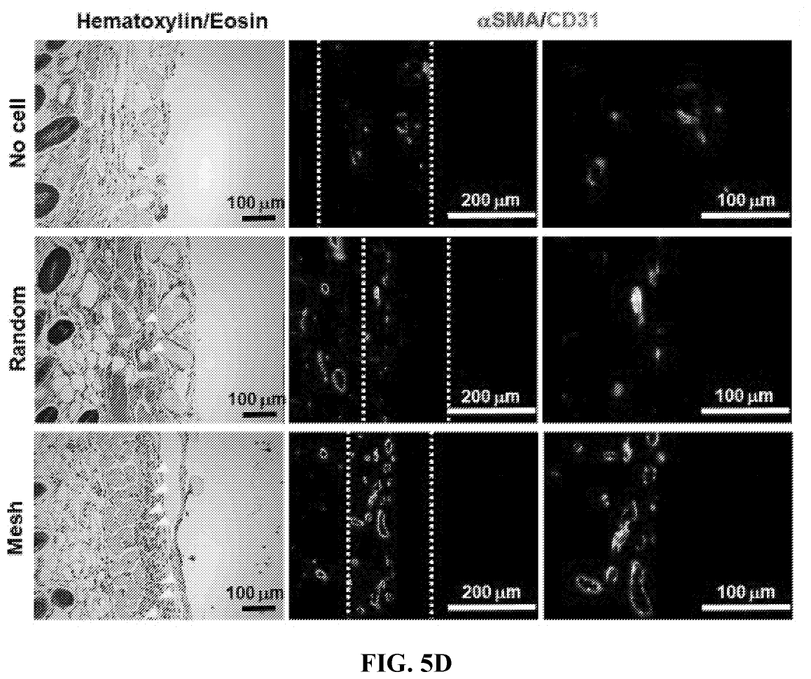

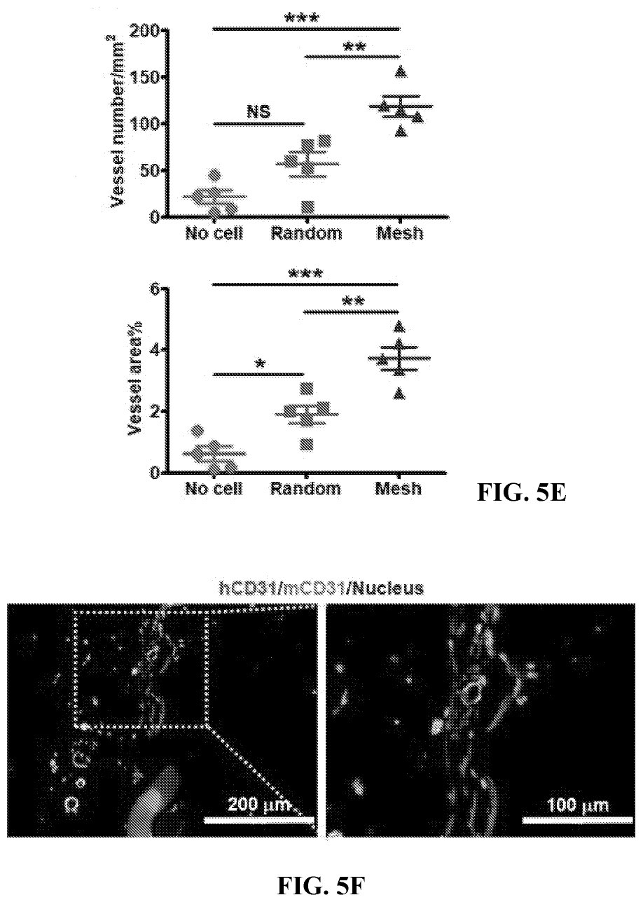

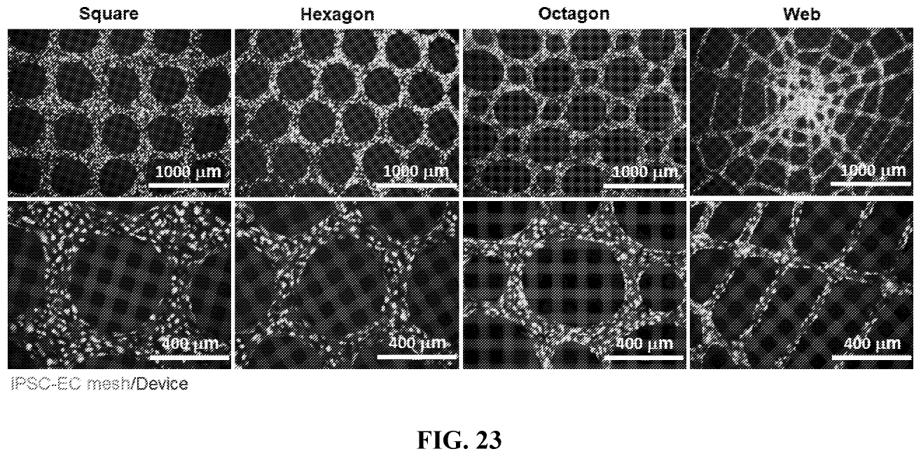

To explore whether the ASA strategy was applicable to other types of ECs and would be potentially used in a clinical setting, human induced pluripotent stem cell-derived endothelial cells (iPSC-ECs) were tested. The iPSC-ECs have been considered as an autologous, unlimited cell source for vascularization (Adams et al., “Functional Vascular Endothelium Derived From Human Induced Pluripotent Stem Cells,” Stem Cell Rep. 1:105-113 (2013), which is hereby incorporated by reference in its entirety), and therefore have great potential for clinical applications. Similar to HUVECs, iPSC-ECs formed various controllable microvascular meshes with fibrin-filled tubular structures on micropillar substrates (FIGS. 5A-5C). The different geometrical meshes can be transferred to diffusion chamber devices (FIG. 23). To evaluate the ability to enhance vascularization, microvascular meshes of square shape were implanted in the subcutaneous space of SCID-Beige mice for 2 weeks. In all in vivo experiments, a small amount of NHDFs were mixed with iPSC-ECs (iPSC-ECs:NHDFs=9:1). As shown in FIG. 5D, H&E staining and immunostaining (CD31 and α-SMA) revealed mature vasculatures at the interface between the device and panniculus carnosus muscle for all three groups. However, the “Mesh” devices (n=5) resulted in significantly more blood vessels in terms of both density and area percentage than the “No cell” (n=5) and “Random” (n=5) devices (FIG. 5E). Moreover, positive staining of human and mouse CD31 antibodies confirmed the anastomoses (FIG. 5F). It was noticed that vascularization ability of iPSC-EC mesh was not as good as HUVEC mesh in terms of the blood vessel density (FIG. 3D and FIG. 5E). Such difference highlights the importance of future research to further optimize the differentiation and vascularization of iPSC-ECs.

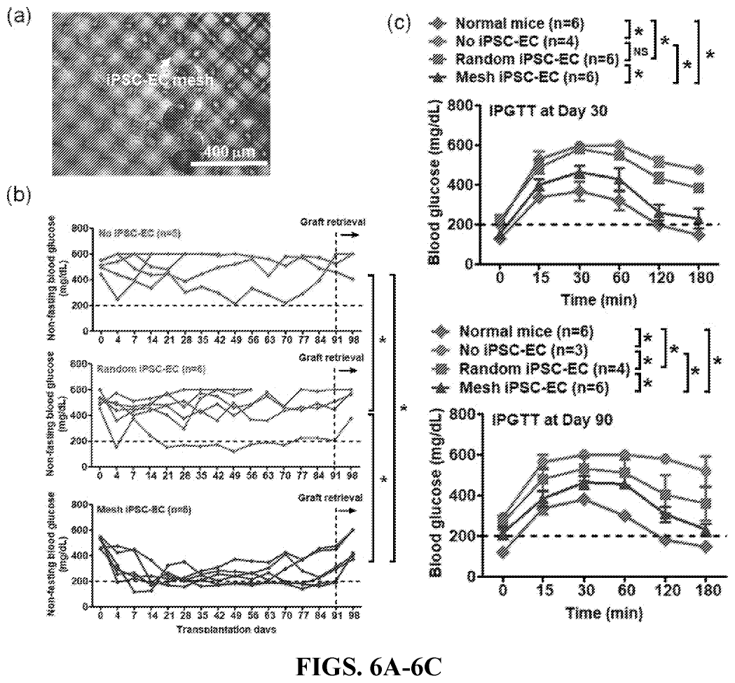

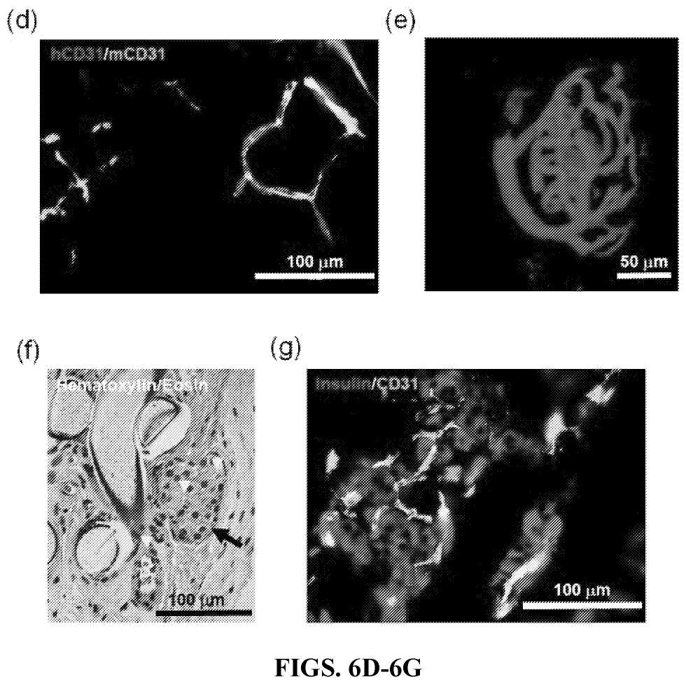

The iPSC-EC mesh was attached to a diffusion chamber device containing rat islets using a fibrin gel (“Mesh iPSC-EC” (n=6); FIG. 6A) similar to HUVEC mesh. The “Mesh” group led to significantly better diabetes correction than the control groups (“No iPSC-EC” (n=5) and “Random iPSC-EC” (n=6)) in the rat-to-mouse transplantation model (FIG. 6B and Table 3). Mice from the “Mesh” group also responded to IPGTTs,

TABLE 3
The Summary of the Number of Cured Mice (Non-Fasting Blood Glucose <200
mg/dL) in No iPSC-EC, Random iPSC-EC, and Mesh iPSC-EC Devices During 91 Days of
Transplantation.

| Transplantation | No iPSC-EC | Random IPSC-EC | Mesh iPSC-EC |
|---|---|---|---|
| days | (cured mice/total mice) | (cured mice/total mice) | (cured mice/total mice |
| 4 | 0/5 | 1/6 | 1/6 |
| 7 | 0/5 | 0/6 | 1/6 |
| 14 | 0/5 (One sick mouse | 0/6 | 4/6 |
| | euthanized) | | |
| 21 | 0/4 | 1/6 | 2/6 |
| 28 | 0/4 | 1/6 | 3/6 |
| 35 | 0/4 | 1/6 | 3/6 |
| 42 | 0/4 (One sick mouse | 1/6 | 3/6 |
| | euthanized) | | |
| 49 | 0/3 | 1/6 | 2/6 |
| 56 | 0/3 | 1/6 (Two sick mice | 2/6 |
| | | euthanized) | |
| 63 | 0/3 | 1/4 | 3/6 |
| 70 | 0/3 | 1/4 | 3/6 |
| 77 | 0/3 | 0/4 | 3/6 |
| 84 | 0/3 | 0/4 | 2/6 |
| 91 | 0/3 (Devices retrieved) | 0/4 (Devices retrieved) | 2/6 (Devices retrieved) |

performed on Day 30 and Day 90, significantly better than those from control groups (FIG. 6C). However, due to the less robust vascularization ability of iPSC-EC mesh, approximately half of mice in “Mesh” group didn't achieve BG correction. Compared to normal mice, the glucose response of mice in “Mesh” group was delayed. In the future, enhancing the function and vascularization of iPSC-EC mesh might help transplanted islets realize improved BG control and glucose responsiveness in IPGTT. In addition, an IPGTT performed immediately after normoglycemia could evaluate the ability of transplanted islets in insulin secretion during glucose stimulation at early period post-transplantation. Immunostaining of the retrieved “Mesh” devices confirmed that iPSC-EC meshes promoted anastomoses between human and mouse vessels (FIG. 6D). Whole mouse perfusion prior to retrieval and confocal imaging showed that the transplanted rat islets were functionally re-vascularized (FIG. 6E). Re-vascularization and insulin secretion by the islets were verified by H&E (FIG. 6F) and immunostaining (FIG. 6G). Blood-perfused vessels were present both inside and outside the islets (H&E image), and the islets were positive for both insulin and CD31 staining. These results demonstrate the feasibility of engineering patient-specific microvascular meshes from iPSC-ECs and the potential use in cell replacement therapies for T1D.

### Discussion of Examples 1-5

Spatially arranged micropillars were utilized to fabricate high resolution, resilient, and transferrable microvascular meshes. Micropillars play two roles in the anchored self-assembly (ASA): guiding the ECs to form patterns with controllable geometry and preventing the cells and matrix from shrinking. Compared to random EC tubes formed on a smooth substrate, the ASA-enabled microvascular meshes are continuous, interconnected, and precisely controlled. In a poorly vascularized subcutaneous space, microvascular meshes promote more efficient vascularization and anastomoses with host vasculature than randomly mixed cells. The subcutaneous space is an attractive transplant site due to its easy accessibility and relatively large capacity for transplantation (Sakata et al., “Strategy for Clinical Setting in Intramuscular and Subcutaneous Islet Transplantation,” Diabetes Metab. Res. Rev. 30:1-10 (2014), which is hereby incorporated by reference in its entirety), however, the subcutaneous space has much less vasculature (Jaap et al., “Skin Capillary Density in Subjects With Impaired Glucose Tolerance and Patients With Type 2 Diabetes,” Diabetic Med. 13:160-164 (1996) and Malik et al., “Skin Epidermal Thickness and Vascular Density in Type 1 Diabetes,” Diabetic Med. 9:263-267 (1992), each of which is hereby incorporated by reference in its entirety) compared to other vascularized sites such as small bowel mesentery (Phelps et al., “Vasculogenic Bio-Synthetic Hydrogel for Enhancement of Pancreatic Islet Engraftment and Function in Type 1 Diabetes,” Biomaterials 34:4602-4611 (2013), which is hereby incorporated by reference in its entirety), omentum (Berman, D. M. et al., “Bioengineering the Endocrine Pancreas: Intraomental Islet Transplantation Within a Biologic Resorbable Scaffold,” Diabetes 65:1350-1361 (2016), which is hereby incorporated by reference in its entirety), and epididymal fat pad (Brady, A. C. et al., “Proangiogenic Hydrogels Within Macroporous Scaffolds Enhance Islet Engraftment in an Extrahepatic Site,” Tissue Eng. Part A 19:2544-2552 (2013), which is hereby incorporated by reference in its entirety). In Random device, cells were randomly dispersed on top of the device, and required time to form an interconnected network. In contrast, microvascular meshes provide a pre-formed, highly interconnected network for secretion of angiogenic factors and generation of vascular sprouts. The hierarchical structures of pre-formed mesh network and newly-branched sprouts efficiently induce ingrowth and anastomoses of host vasculature. Likely due to such multilevel configuration, microvascular meshes resulted in a high vasculature density. In this study, the focus was on the square microvascular mesh, with its vascularization compared with random cells on the smooth substrate without any micropillars. Given that a square shape might not be optimal for all purposes, future research will compare the effects of different microvascular mesh openings (controlled by micropillar size/interval) and geometries (controlled by micropillar arrangement) on the vascularization. The SCID-Beige mice were chosen as recipients to avoid the compounding effect of immune rejections, similar to most previous studies (Pepper, A. R. et al., “A Prevascularized Subcutaneous Device-Less Site for Islet and Cellular Transplantation,” Nat. Biotechnol. 33:518-523 (2015); Vlahos et al., “Modular Tissue Engineering for the Vascularization of Subcutaneously Transplanted Pancreatic Islets,” Proc. Natl. Acad. Sci. U.S.A. 114:9337-9342 (2017); and Weaver, J. D. et al., “Vasculogenic Hydrogel Enhances Islet Survival, Engraftment, and Function in Leading Extrahepatic Sites,” Sci. Adv. 3:e1700184 (2017), each of which is hereby incorporated by reference in its entirety). However, this strain is also known to promote angiogenesis. Immunocompetent animals and autologous endothelial and supporting cells should be tested in the future to show the translational potential of this approach.

The resilient and transferrable microvascular meshes that we described in this work make it possible to vascularize islets subcutaneously transplanted in retrievable delivery devices. Cell replacement therapy such as intrahepatic transplantation of donor islets is relatively successful in some patients. However, there are a number of limitations including immediate blood-mediated inflammatory response (Veriter et al., “Bioengineered Sites for Islet Cell Transplantation,” Curr. Diabetes Rep. 13:745-755 (2013), which is hereby incorporated by reference in its entirety), potential risks of thrombosis (Ryan et al., “Successful Islet Transplantation: Continued Insulin Reserve Provides Long-Term Glycemic Control,” Diabetes 51:2148-2157 (2002), which is hereby incorporated by reference in its entirety), and localized steatosis (Markmann et al., “Magnetic Resonance-Defined Periportal Steatosis Following Intraportal Islet Transplantation: A Functional Footprint of Islet Graft Survival?,” Diabetes 52:1591-1594 (2003), which is hereby incorporated by reference in its entirety), and inability to retrieve or replace failed cells (An et al., “Designing a Retrievable and Scalable Cell Encapsulation Device for Potential Treatment of Type 1 Diabetes,” Proc. Natl. Acad. Sci. U.S.A. 115:E263-E272 (2018), which is hereby incorporated by reference in its entirety). Therefore, great efforts have been made to find an alternative islet transplantation strategy that is less invasive, supports long-term cell function, and allows cell retrieval or replacement (Pepper et al., “A Prevascularized Subcutaneous Device-Less Site for Islet and Cellular Transplantation,” Nat. Biotechnol. 33:518-523 (2015); Weaver et al., “Vasculogenic Hydrogel Enhances Islet Survival, Engraftment, and Function in Leading Extrahepatic Sites,” Sci. Adv. 3:e1700184 (2017); An et al., “Designing a Retrievable and Scalable Cell Encapsulation Device for Potential Treatment of Type 1 Diabetes,” Proc. Natl. Acad. Sci. U.S.A. 115:E263-E272 (2018); An et al., “Developing Robust, Hydrogel-Based, Nanofiber-Enabled Encapsulation Devices (NEEDs) for Cell Therapies,” Biomaterials 37:40-48 (2015); Veiseh et al., “Size- and Shape-Dependent Foreign Body Immune Response to Materials Implanted in Rodents and Non-Human Primates,” Nat. Mater. 14:643-651 (2015); and Baidal et al., “Bioengineering of an Intraabdominal Endocrine Pancreas,” N. Engl. J. Med. 376:1887-1889 (2017), each of which is hereby incorporated by reference in its entirety). Previous studies have shown that rat islets subcutaneously transplanted in immunodeficient mice maintained short-term normoglycemia (˜20 to 28 days) at the low dose (˜375 to 750 islets or IEQ), but the long-term islet function was not presented (Vlahos et al., “Modular Tissue Engineering for the Vascularization of Subcutaneously Transplanted Pancreatic Islets,” Proc. Natl. Acad. Sci. U.S.A. 114:9337-9342 (2017) and Sorenby et al., “Preimplantation of an Immunoprotective Device Can Lower the Curative Dose of Islets to That of Free Islet Transplantation-Studies in a Rodent Model,” Transplantation 86:364-366 (2008), each of which is incorporated by reference in its entirety). The Examples of the present application demonstrate that the microvascular meshes significantly improved the function of rat islets (approximate 500 IEQ) in the poorly vascularized but convenient subcutaneous space and enabled diabetes correction in SCID-Beige mice for up to 3 months. Importantly, given that microvascular meshes may be made from autologous cells such as iPSC-ECs and can be transferred to different delivery or immuno-protective devices, this approach may contribute to an immunosuppression-free cell replacement therapy for T1D.

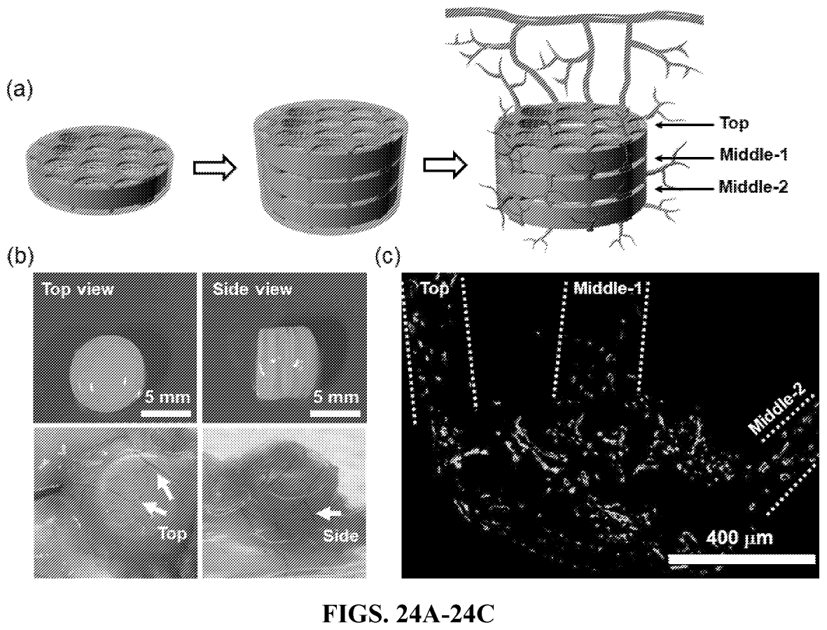

The scalability is an important requirement for cell replacement therapies and has been challenging for subcutaneous devices (An et al., “Designing a Retrievable and Scalable Cell Encapsulation Device for Potential Treatment of Type 1 Diabetes,” Proc. Natl. Acad. Sci. U.S.A. 115:E263-E272 (2018); Desai et al., “Advances in Islet Encapsulation Technologies,” Nat. Rev. Drug Discovery 16:367 (2017); Scharp et al., “Encapsulated Islets for Diabetes Therapy: History, Current Progress, and Critical Issues Requiring Solution,” Adv. Drug Delivery Rev. 67-68:35-73 (2014); and Smink et al., “Toward Engineering a Novel Transplantation Site for Human Pancreatic Islets,” Diabetes 62:1357-1364 (2013), each of which is hereby incorporated by reference in its entirety). ASA-enabled microvascular meshes can be fabricated in larger sizes (e.g. ˜25 cm²) and stacked with alternating devices in the z direction for scale up (FIG. 24). High level of vascularization was observed in stacked and thick construct consisting of three alternating layers of microvascular meshes and devices. Given that different types of ECs may be isolated from the body (Rafii et al., “Isolation and Characterization of Human Bone Marrow Microvascular Endothelial Cells: Hematopoietic Progenitor Cell Adhesion,” Blood 84:10-19 (1994); Kern et al., “Isolation and Culture of Microvascular Endothelium From Human Adipose Tissue,” J. Clin. Invest. 71:1822-1829 (1983); and Hirschi et al., “Assessing Identity, Phenotype, and Fate of Endothelial Progenitor Cells,” Arterioscler., Thromb., Vasc. Biol. 28:1584-1595 (2008), each of which is hereby incorporated by reference in its entirety), patient-specific and tissue/organ-specific microvascular meshes could be created using the ASA. Moreover, microvascular meshes may be combined with other cell types such as hepatocytes and cardiomyocytes for liver and cardiovascular engineering applications, or fibroblasts and smooth muscle cells to assist wound healing. Lastly, the ASA as a general approach may be expanded to other bioengineering fields to construct geometrically defined, high resolution live materials at micro-scales, or to organize therapeutic cells into specific patterns for diverse applications.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A vascularized therapeutic delivery system, said system comprising:
a preparation of cells encapsulated by a biological support material and
a microvascular mesh that at least partially surrounds the biological support material encapsulating the preparation of cells, wherein the microvascular mesh comprises a network of continuous interconnected tubular structures defined by endothelial cells and an extracellular matrix scaffold.

2. The vascularized therapeutic delivery system of claim 1, wherein the preparation of cells is a preparation of any one or more of endothelial cells, smooth muscle cells, cardiac muscle cells, cardiac myocytes, epithelial cells, urothelial cells, fibroblasts, myoblasts, chondrocytes, chondroblasts, osteoblasts, keratinocytes, hepatocytes, renal cells, pulmonary cells, bile duct cells, pancreatic islet cells, thyroid cells, parathyroid cells, adrenal cells, hypothalamic cells, pituitary cells, ovarian cells, testicular cells, salivary gland cells, adipocytes, embryonic stem cells, adult stem cells, induced pluripotent stem cells, mesenchymal stem cells, neuronal cells, astrocytes, oligodendrocytes, hematopoietic cells, and any precursor or progenitor cell thereof.

3. The vascularized therapeutic delivery system of claim 1 or claim 2, wherein the preparation of cells is a preparation of cells engineered to recombinantly express a therapeutic agent.

4. The vascularized therapeutic delivery system of any one of claims 1-3, wherein the preparation of cells is a preparation of pancreatic islet cells.

5. The vascularized therapeutic delivery system of any one of claims 1-4, wherein the biological support material is a synthetic polymer material selected from the group consisting of polycaprolactone, polylactic acid, polyglycolide, poly(lactic-co-glycolic) acid, polytetrafluoroethylene, nylon, polydimethylsiloxane, polyvinylchloride, polyvinylidene fluoride, polyurethane isocyanates, alginate, cellulose acetate, cellulose nitrate, polysulfone, polyether sulfones, polystyrene, polyurethane, polyvinyl alcohols, polyvinylidenes, polyvinyl chloride copolymers, polyacrylonitrile, poly(acrylonitrile/covinyl chloride), polyamides, polymethylmethacrylate, polyacrylates, polyphosphazenes, polyethylene oxide, and mixtures thereof.

6. The vascularized therapeutic delivery system of any one of claims 1-5, wherein the biological support material is a material selected from the group consisting of hydrogel, collagen, hyaluronate, fibrin, fibroin, alginate, agarose, chitosan, bacterial cellulose, elastin, keratin, and combinations thereof.

7. The vascularized therapeutic delivery system of any one of claims 1-7, wherein the extracellular matrix scaffold is comprised of an extracellular matrix component selected from fibronectin, laminin, heparin, collagen, glycosaminoglycan, proteoglycan, elastin, fibrin, fibroin, and combinations thereof.

8. The vascularized therapeutic delivery system of claim 7, wherein the extracellular matrix scaffold comprises fibrin.

9. The vascularized therapeutic delivery system of any one of claims 1-8, wherein the tubular structures of the microvascular mesh have a diameter of about 15 μm to about 200 μm.

10. The vascularized therapeutic delivery system of any one of claims 1-9, wherein the tubular structures of the microvascular mesh are further defined by one or more type of support cell.

11. The vascularized therapeutic delivery system of claim 10, wherein the one or more type of support cell is selected from fibroblasts, smooth muscle cells, mesenchymal stem cells, and perivascular cells.

12. The vascularized therapeutic delivery system of any one of claims 1-11, wherein one or more of the tubular structures are filled with extracellular matrix scaffold.

13. The vascularized therapeutic delivery system of any one of claims 1-11 wherein one or more of the tubular structures comprise a lumen.

14. The vascularized therapeutic delivery system of any one of claims 1-13, wherein the endothelial cells are human umbilical vein endothelial cells (HUVEC).

15. The vascularized therapeutic delivery system of any one of claims 1-14, wherein the endothelial cells are derived from induced pluripotent stem cells.

16. The vascularized therapeutic delivery system of any one of claims 1-15, wherein the microvascular mesh has a plurality of openings, wherein each opening in the mesh ranges in size from about 20 μm to about 1000 μm.

17. The vascularized therapeutic delivery system of any one of claims 1-16, wherein the microvascular mesh has a density of openings ranging from 1 to 100 openings/mm².

18. The vascularized therapeutic delivery system of any one of claims 1-17, wherein mesh openings have a triangular, quadrilateral, pentagon, hexagon, heptagon, octagon, nonagon, decagon, circular, or microcapillary geometry.

19. The vascularize therapeutic delivery system of any one of claim 1-17, wherein the network of tubular structures and openings of the microvascular mesh are organized in a web pattern.

20. A method for delivering a therapeutic agent to a subject, said method comprising:
selecting a subject in need of a therapeutic agent;
providing the vascularized therapeutic delivery system of any one of claims 1-19; and
implanting the vascularized therapeutic delivery system into a region of the selected subject suitable for delivering the therapeutic agent.

21. The method of claim 20, wherein the vascularized therapeutic delivery system is implanted into a subcutaneous region of the selected subject.

22. The method of claim 20, wherein the therapeutic agent comprises the preparation of encapsulated cells of the vascularized therapeutic delivery system.

23. The method claim 20, wherein the therapeutic agent is released from the preparation of encapsulated cells of the vascularized therapeutic delivery system.

24. The method of claim 20, wherein the microvascular mesh of the vascularized therapeutic delivery system comprises endothelial cells obtained from the selected subject.

25. The method of claim 20, wherein the subject is a human.

26. The method of claim 20 further comprising:
retrieving and replacing the vascularized therapeutic delivery system in the selected subject.

27. A method of producing a vascularized therapeutic delivery system, said method comprising:
providing a monolithic substrate comprising a planar surface interrupted by a plurality of micropillars;
applying endothelial cells suspended in an extracellular matrix material to the planar surface of the monolithic substrate;
culturing the suspended endothelial cells under conditions effective for the endothelial cells to organized into a microvascular mesh, wherein said microvascular mesh comprises a network of continuous interconnected tubular structures defined by the endothelial cells and extracellular matrix material;
transferring the microvascular mesh to an outer surface of an implantable cell delivery construct comprising a preparation of cells, thereby producing the vascularized therapeutic delivery system.

28. The method of claim 27, wherein the microvascular mesh has openings, wherein said openings in the mesh range in size from 20 μm to 1000 μm.

29. The method of claim 27 or claim 28, wherein the microvascular mesh has a density of openings ranging from 1 to 100 openings/mm².

30. The method of any one of claims 27-29, wherein the micropillars are sized and spatially arranged on the surface of the substrate in a manner effective to define mesh opening geometry.

31. The method of any one of claim 30, wherein the mesh openings have a triangular, quadrilateral, pentagon, hexagon, heptagon, octagon, nonagon, decagon, circular, or microcapillary geometry.

32. The method of any one of claims 27-30, wherein the network of tubular structures and openings of the microvascular mesh are organized in a web pattern.

33. The method of any one of claims 27-32, wherein each of the plurality of micropillars has a diameter of about 100 μm to about 400 μm.

34. The method of any one of claims 27-33, wherein distance between adjacent micropillars on the surface of the substrate is about 50 μm to about 500 μm.

35. The method of any one of claims 27-34, wherein of the tubular structures of the microvascular mesh have an internal diameter of about 15 μm to about 200 μm.

36. The method of any one of claims 27-35, wherein the endothelial cells are human umbilical vein endothelial cells (HUVEC).

37. The method any one of claims 27-36, wherein the endothelial cells are derived from induced pluripotent stem cells.

38. The method of any one of claims 27-37, wherein one or more type of support cell is suspended along with the endothelial cells in the extracellular matrix material for said applying, and said tubular structures are further defined by said one or more type of support cell.

39. The method of claim 38, wherein the one or more type of support cell is selected from fibroblasts, mesenchymal stem cells, smooth muscle cells, and perivascular cells.

40. The method of any one of claims 27-39, wherein the extracellular matrix material is comprised of an extracellular matrix component selected from fibronectin, laminin, heparin, collagen, glycosaminoglycan, proteoglycan, elastin, fibrin, fibroin, and combinations thereof.

41. The method of any one of claims 27-40, wherein the preparation of cells in the implantable cell delivery construct is a preparation of any one or more of smooth muscle cells, endothelial cells, cardiac muscle cells, cardiac myocytes, epithelial cells, urothelial cells, fibroblasts, myoblasts, chondrocytes, chondroblasts, osteoblasts, keratinocytes, hepatocytes, bile duct cells, pancreatic islet cells, thyroid cells, parathyroid cells, adrenal cells, hypothalamic cells, pituitary cells, ovarian cells, testicular cells, salivary gland cells, adipocytes, embryonic stem cells, induced pluripotent stem cells, adult stem cells, mesenchymal stem cells, neural cells, astrocytes, oligodendrocytes, hematopoietic cells, and any precursor or progenitor cell thereof.

42. The method of any one of claims 27-41, wherein the preparation of cells in the implantable cell delivery construct is a preparation of cells engineered to recombinantly express a therapeutic agent.

43. The method of any one of claims 27-42 further comprising:
stacking two or more vascularized therapeutic delivery systems together vertically and
encapsulating said stacked vascularized therapeutic delivery systems in an angiogenic support material.

44. The method of claim 43, wherein the angiogenic support material is selected from hydrogel, collagen, hyaluronate, fibrin, fibroin, alginate, agarose, chitosan, bacterial cellulose, elastin, keratin, and combinations thereof.

45. The method of any one of claims 27-44 further comprising:
selecting a subject in need of a therapeutic agent and
implanting the vascularized therapeutic delivery system into said subject.
